(19) United States
(12) Patent Application Publication
(10) Pub. No.: US 2005/0227356 A1
(43) Pub. Date: Oct. 13, 2005

(54) Novel compositions and methods for genetic manipulation of Rhodococcus bacteria

(76) Inventors: Philip A. Lessard, Framingham, MA (US); Anthony J. Sinskey, Boston, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/820,451

(22) Filed: Apr. 7, 2004

(51) Int. Cl. C12Q 1/68; C12N 15/74; C12N 1/21

(52) U.S. Cl. 435/476; 435/6; 435/252.3

(57) ABSTRACT

The present invention provides novel compostions and methods of using the same for genetic manipulation of a variety of bacterial strains such as Rhodococcus.

### BACKGROUND OF THE INVENTION

Gram-positive bacteria belonging to the genus Rhodococcus, some of which were formerly classified as Nocardia, Mycobacterium, Gordona, or Jensenia spp., or as members of the “rhodochrous” complex, are widely distributed in the environment. Members of the genus. Rhodococcus exhibit a wide range of metabolic activities, including antibiotic and amino acid production, biosurfactant production, and biodegradation and biotransformation of a large variety of organic and xenobiotic compounds. Due to these diverse enzymatic activities and a demonstrated solvent tolerance, rhodococci have many practical applications for bioconversion and bioremediation. Unfortunately, few appropriate genetic tools exist to investigate and exploit these metabolic activities in Rhodococcus and like organisms.

One hindrance to the full exploitation of Rhodococcus is the dearth of genetic tools available for strain manipulation. Among the most basic of tools for genetic manipulation of bacteria are plasmids. Plasmids can be used to introduce new or extra copies of genes into bacterial cells and to complement mutations in bacteria. They can be used to manipulate expression levels of different gene products, impacting a variety of cellular processes. Plasmids can also be used to deliver transposable elements or as intermediates in knocking out gene function via homologous recombination. Plasmids also play an important role in horizontal gene transfer among bacteria.

The lack of plasmid vectors suitable for use in Rhodococcus has led several groups to develop binary vectors, in which an extant Rhodococcus plasmid is combined with an Escherichia coli plasmid forming a new vector that replicates in both cell types. A limited number of shuttle vectors have been developed for Rhodococcus based on this principle. However it is difficult or nearly impossible to generate transformants with the majority of these. This and other evidence (supra) suggests that the rather narrow host ranges of the currently known shuttle vectors limits their use to only a few Rhodococcus species. To overcome this problem we sought to develop a new plasmid vector specifically for working with a variety of Rhodococcus strains.

### SUMMARY OF THE INVENTION

The present invention provides novel nucleic acids and vectors comprising such nucleic acids for the manipulation of genes in Rhodococcus and other members of the Actinomycetales bacterial family.

In certain embodiments, the isolated nucleic acids sequences of the invention support replication in Rhodococcus and may comprise a nucleic acid sequence selected from the group consisting of: (a) SEQ ID NO: 2; (b) a nucleic acid sequence that hybridizes with (a) under stringent hybridization conditions; and (c) a nucleic acid sequence that is complementary to (a) or (b). In other embodiments, such isolated nucleic acids may comprise a nucleic acid sequence selected from the group consisting of: (a) SEQ ID NO: 5; (b) SEQ ID NO: 6; (c) SEQ ID NO: 4; (d) a sequence that hybridizes with (a), (b), or (c) under stringent hybridization conditions; and (e) a nucleic acid sequence that is complementary to (a), (b), (c), or (d). In still other embodiments, such nucleic acids may comprise a nucleic acid sequence selected from the group consisting of: (a) a nucleic acid sequence comprising SEQ ID NOs: 4, 5, and 6; (b) a nucleic acid sequence comprising SEQ ID NOs: 5 and 6; (c) a nucleic acid sequence comprising SEQ ID NOs: 4 and 5; (d) a nucleic acid sequence comprising SEQ ID NOs: 4 and 6; (e) a nucleic acid sequence that hybridizes with (a), (b), (c) or (d) under stringent hybridization conditions; and (f) a nucleic acid sequence that is complementary to (a), (b), (c), (d), or (e). In certain embodiments, the isolated nucleic acids may support conditional replication, i.e. replication that is temperature sensitive.

The present invention also provides methods for identifying plasmids with properties of temperature sensitivity in Rhodococcus. For example, such a method may comprise: (a) isolating DNA from a Rhodococcus bacterium that cannot grow well above a minimum temperature; (b) examining the DNA for the presence of small plasmids; (c) extracting the small plasmids; (d) testing the small plasmids for temperature sensitive replication in Rhodococcus strains that grow above the minimum temperature.

In other embodiments, the isolated nucleic acids of the invention are necessary for conjugative transfer of a plasmid in one Rhodococcus bacterium to another bacterium. In certain embodiments, such isolated nucleic acids may comprise a nucleic acid sequence selected from the group consisting of: (a) SEQ ID NO: 3; (b) a nucleic acid sequence that hybridizes with (a) under stringent hybridization conditions; and (c) an isolated nucleic acid that is complementary to (a) or (b). In other embodiments, such isolated nucleic acids may comprise a nucleic acid sequence selected from the group consisting of: (a) the origin of Transfer (oriT) sequence; (b) a nucleic acid sequence that hybridizes with (a) under stringent hybridization conditions; and (c) a nucleic acid sequence that is complementary to (a) or (b). In certain embodiments, a nucleic acid sequence that enables conjugative transfer of the plasmid may comprise a nucleic acid selected from the group consisting of: (a) a nucleic acid sequence comprising the region involved in conjugal transfer containing origin of Transfer (oriT) sequence; (b) a nucleic acid sequence comprising the origin of Transfer (oriT) sequence; (c) a nucleic acid sequence that hybridizes with (a) or (b) under stringent hybridization conditions; and (d) a nucleic acid sequence that is complementary to (a), (b) or (c).

The present invention also provides methods for identifying plasmids with the ability to conjugatively transfer from one Rhodococcus bacterium to another bacterium. For example, such methods may comprise: (a) isolating DNA from a Rhodococcus bacterium; (b) examining the DNA for the presence of small plasmids; (c) extracting the small plasmids; (d) transforming a Rhodococcus bacterium with the small plasmid; (e) culturing the transformed Rhodococcus bacterium of (d) for a length of time and under conditions with another bacterium, such that conjugative transfer is facilitated, wherein if conjugative transfer is observed, the small plasmid has the ability to conjugatively transfer.

The present invention also provides plasmids comprising at least one of the above described isolated nucleic acids. In certain embodiments, the plasmids of the invention comprise a nucleic acid sequence that supports replication and a nucleic acid sequence that is necessary for conjugative transfer of a plasmid in one Rhodococcus bacterium to another bacterium. In certain embodiments, the replication of the plasmid is temperature-sensitive. For example, in certain plasmids, the ability to replicate of the plasmid is reduced at temperatures above 30° C. In certain embodiments, the plasmid may comprise SEQ ID NO: 1. Such plasmid may enable conditional replication of the plasmid in a donor cell and is able to be transmitted via conjugative transfer to another cell.

Such plasmids may be modified for gene expression in a Rhodococcus strain. For example, the plamid may further comprise gene constructs comprising at least one promoter suitable for the expression of a gene in a Rhodococcus strain and a nucleic acid sequence encoding a gene to be expressed. The present invention also provides methods of using such plasmids in a method for the expression of a nucleic acid in a Rhodococcus bacterium. For example, such a method may comprise: a) providing a plasmid of the invention modified for the expression of a gene; b) transforming a Rhodococcus bacterium with the plasmid of (a); and c) culturing the transformed Rhodococcus bacterium of (b) for a length of time and under conditions whereby the nucleic acid sequence encoding a gene to be expressed is expressed.

The present invention further provides a transformed bacterium comprising any of the above-described plasmids. Such bacterium may be of the genus Rhodococcus, for example, one selected from the group including, but not limited to, Rhodococcus equi, Rhodococcus erythropolis, Rhodococcus opacus, Rhodococcus rhodochrous, Rhodococcus globerulus, Rhodococcus koreensis, Rhodococcus fascians, Rhodococcus I24, Rhodococcus KY1, Rhodococcus B264-1, and Rhodococcus ruber. However, any bacterium of the Actinomycetales bacterial family may comprise a plasmid of the present invention.

The present invention also provides methods for controlling replication of a temperature sensitive plasmid in a Rhodococcus bacterium. For example, such a method may comprise: a) providing a plasmid that is unable to replicate above a certain temperature; b) transforming a Rhodococcus bacterium with the plasmid of (a); and c) controlling the replication of the plasmid by adjusting the temperature of the culture. In certain embodiments, the replication is inhibited by elevating the temperature.

The present invention also provides methods for effecting conjugative transfer of plasmids between Rhodococcus strains. For example, such a method may comprise: a) providing a plasmid with the ability to conjugatively transfer from one Rhodococcus bacterium to another bacterium; b) transforming a Rhodococcus bacterium with the plasmid of (a); and c) culturing the transformed Rhodococcus bacterium of (b) for a length of time and under conditions with another Rhodococcus bacterium such that conjugative transfer occurs.

Still further, the present invention provides methods for manipulating genes in Rhodococcus and other bacteria. For example, in certain embodiments, a method for introducing a nucleic acid into a Rhodococcus bacterium may comprise culturing a first strain of Rhodococcus bacterium transformed with a plasmid comprising a nucleic acid sequence that enables conjugative transfer of the plasmid with a second strain of Rhodococcus bacterium for a length of time under conditions such that conjugative transfer occurs between the first and second strains of bacterium, whereby the plasmid is transferred from the first strain of bacterium to the second strain of bacterium. In certain embodiments, the plasmid may further comprise a gene construct, for example, encoding a protein to be expressed in the second strain of bacterium. Such gene construct may further comprise at least one promoter suitable for the expression of a gene in a Rhodococcus bacterium. The gene may be expressed in the second strain of Rhodococcus bacterium following the transfer. In certain embodiments, the plasmid may comprise a mutant gene or a fragment of a gene, e.g. to use in gene disruption or knock-out. In certain embodiments, the plasmid or a portion thereof may be integrated into the genome of the second strain of bacterium, for example, by homologous recombination. In certain embodiments, the replication of the plasmid may be temperature sensitive, and the plasmid may comprise (a) SEQ ID NO: 2; (b) a sequence that hybridizes with (a) under stringent hybridization conditions; and (c) a sequence that is complementary to (a) or (b). In methods wherein the plasmid replication is temperature sensitive, the method may further comprise the step of selecting a bacterium of the second strain that has integrated the plasmid or a portion thereof into its genome by elevating the temperature of the culture.

In one embodiment, the present invention provides a method for disrupting or knocking out a gene in a Rhodococcus bacterium, comprising culturing a first strain of Rhodococcus bacterium transformed with a plasmid comprising a nucleic acid sequence that enables conjugative transfer of the plasmid and a nucleic acid sequence that serves to disrupt or knock out the gene with a second strain of Rhodococcus bacterium for a length of time under conditions such that conjugative transfer occurs between the first and second strains of bacterium, whereby the plasmid is transferred from the first strain of bacterium to the second strain of bacterium and the sequence that serves to disrupt or knock out the gene is integrated into the genome of the second second strain of bacterium. Such integration may occur by homologous recombination.

Still further, the present invention provides methods for detecting homologous recombination in or selecting from a culture a recipient Rhodococcus bacterium that has integrated a plasmid or a portion thereof into its genome comprising the use of temperature selection. In certain embodiments, such methods may comprise conjugatively transferring from a donor Rhodococcus bacterium to a recipient Rhodococcus bacterium that can grow above a minimum temperature a plasmid that cannot replicate above the minimum temperature; elevating the temperature of the culture above the minimum temperature for a sufficient time to prevent replication of the plasmid and survival of the donor Rhodococcus bacterium, whereby only a recipient Rhodococcus bacterium that has incorporated the plasmid into its genome survives such temperature elevation. In certain embodiments of these methods, the plasmid may comprises (a) the minimal replicon sequence; (b) a sequence that hybridizes with (a) under stringent hybridization conditions; and (c) a sequence that is complementary to (a) or (b). In some embodiments, the minimum temperature in 30° C. and the temperature is elevated to 37° C. In some embodiments, the donor Rhodococcus bacterium is of the strain Rhodococcus B264-1 and the recipient Rhodococcus bacterium is of the strain Rhodococcus I24.

The present invention also provides kits for gene expression and manipulation in Rhodococcus and other bacteria. In certain embodiments, the kit comprises an isolated nucleic acid of the invention. In certain embodiments, the nucleic acid comprises a plasmid of the invention. In yet other embodiments, the kit may comprise a culture of transformed bacteria of the present invention.

### BRIEF DESCRIPTION OF THE DRAWINGS

FIG. 1 shows an analysis of genomic DNA from Rhodococcus sp. B264-1. (A) pB264 can be observed among total genomic DNA from Rhodococcus sp. B264-1. U, undigested genomic DNA (and RNA); E, EcoRI digest of genomic DNA; H, HindIII digest of genomic DNA; B, BamHI digest of genomic DNA; M, BstEII digest of lambda DNA. (B) Pulsed field gel electrophoresis of DNA from Rhodococcus. L, lambda ladder (˜50 kb) molecular weight marker; B, genomic DNA from Rhodococcus sp. B264-1. In addition to the chromosomal DNA, which migrates near the top of the marker DNA, two additional bands can be seen in the 250-300 kb range. Additional material migrates at less then 50 kb, although resolution of the gel in this size range is not reliable under the conditions used; S, genomic DNA from R. erythropolis SQ1. For both panels, the sizes of key molecular weight markers are indicated in kb.

FIG. 2 shows key plasmids used in this invention. pB264 is depicted as it occurs in Rhodococcus sp. B264-1. pAL298 is a binary plasmid that carries both pB264 (indicated in yellow) and a ColEI-origin of replication, as well as GntR (aacC1) and AmpR markers. The ColEI origin does not by itself support replication in Rhodococcus sp. strains B264-1 or I24 (not shown). pJANET, contains the bulk of pB264 in conjunction with a GntR marker and an origin of replication derived from pNG2 [21]. Note that the pNG2 origin replicates well in both E. coli and Rhodococcus and that it functions well at both 30° C. and 37° C. The relative positions and strandedness of each of the 9 ORFs within pB264 are indicated as heavy black lines.

FIG. 3 shows deletion derivatives of pB264 used to identify domains involved in (A and B) replication and (C) conjugal transfer. The linear representation of the pB264 element indicates relative positions of the restriction sites for XhoI (X), SacII (Sc), and SphI (Sp), as well as the positions and orientations of ORFs 1 through 9. (A) Deletion derivatives of pB264 that retain the ability to replicate in Rhodococcus. The entire pB264 element is represented in the plasmid pAL298. For this plasmid and all derivatives of pAL298, only the portions derived from pB264 are shown (as black boxes), while deleted regions are represented as dotted lines. Note that in plasmid 2 (and all derivatives of plasmid 2), the region bounded by the two SacII sites denoted by asterisks (*) was inverted during plasmid construction. Plasmids 1-8 could all be propagated by themselves in Rhodococcus sp. I24. Plasmid 9, however, could only be maintained in Rhodococcus sp. I24 when either pAL312 or pAL314 were present. Plasmids 10 and 11 could only be maintained in Rhodococcus sp. I24 when pAL314 was present. (B) Regions of pB264 supplied in trans to support replication of plasmids 9, 10, and 11. (C) Deletion derivatives of pB264 that retain the origin of conjugal transfer. Plasmid 16 (pJANET) contains the bulk of the pB264 element, lacking only a 136 bp fragment at the 5′ end. While pAL281 can not be transferred from Rhodococcus sp. B264-1 to other rhodococci via conjugation, plasmids 15 through 20 are readily transferred.

FIG. 4 show a summary of structural elements within regions of pB264 involved in replication and conjugal transfer. (A) Schematic representation of pB264, showing relative positions of restriction sites (as in FIG. 3), as well as the positions and relative orientations of nine open reading frames (long arrows) and several repeat elements (small arrowheads). ORFs for which a function has been demonstrated are indicated in darker blue. Numbering of the repeat elements is as defined by Kulakov et al. [16]. Numbers in italics indicate single copies of inverted repeat elements, while the paired elements within the direct repeats are indicated by a single label (DR1 or DR2). Below the diagram of pB264 are schematic representations of the regions involved in replication of pB264 [defined as the intersection of the two smallest autonomously replicating plasmids (plasmids 6 and 8) described in FIG. 3] and conjugation of pB264 [defined as the intersection of the two smallest conjugative plasmids (18 and 20) described in FIG. 3]. Note that only 9 of the 14 nt comprising one copy of inverted repeat 2 are retained within the replication region (far left). (B) Sequence detail of the region near DR1. The direct repeat is composed of two 30 nt elements (DR1a and DR1b) indicated in uppercase letters. This region also possesses four smaller (16 nt) direct repeat elements, indicated by arrows above the sequence. (C) Sequence detail in the region of DR2. The two repeat elements that comprise DR2 (DR2a and DR2b) are indicated in uppercase letters. A sequence element that is conserved among pAL5000-type plasmids is nested within the repeat elements (underlined). Note that the smallest autonomously replicating plasmid bears only DR2a and a single copy of the pAL5000 element and extends only to the position marked “deletion terminus.”

FIG. 5 shows the full length sequence of pB264 (GenBank accession AY297818) (SEQ ID NO:1).

FIG. 6 corresponds to the nucleic acid sequence of pB264 capable of autonomous replication in Rhodococcus (SEQ ID NO:2).

FIG. 7 corresponds to the nucleic acid sequence of pB264 involved in conjugal transfer, which includes an origin of Transfer (oriT) (SEQ ID NO: 3).

FIG. 8 is the oriV sequence of pB264 (SEQ ID NO: 4).

FIG. 9 corresponds to the nucleic acid sequence of ORF7 of pB264 (SEQ ID NO:5). ORF7 is also corresponds to the coding sequence of the repA gene, which encodes RepA.

FIG. 10 corresponds to the nucleic acid sequence of ORF8 of pB264 (SEQ ID NO:6). ORF8 is also corresponds to the coding sequence of the repB gene, which encodes RepB.

FIG. 11 depicts the pAL302 plasmid.

FIG. 12 depicts the nimA knock out strategy, in which pAL302 is inserted into the KY1 genome by homologous recombination. The white arrow corresponds to the nimA gene; purple arrow to the internal nimA fragment (nimA *); and the brown arrow to the gentamicin resistence marker (aacC1).

FIG. 13 depicts the PCR primer localization in the wildtype genome (KO1 and KO2); in pAL302 (KO3 and KO4); and after insertion of pAL302 into the KY1 genome by homologous recombination.

FIG. 14 depicts the experimental strategy to eliminate wildtype nimA.

FIG. 15 depicts the location of the restriction sites for Cla I and SnaB I in the wildtype genome and the recombinant genome; pAL302 does not contain restriction sites for Cla I or SnaB I.

FIG. 16 shows the indene metabolism profile of Rhodococcus sp. KY1 induced by indene (final concentration 2 mM).

FIG. 17 shows indene metabolism profile of the mutant strain 7++ induced by indene (final concentration 2 mM).

FIG. 18 shows indene metabolism profile of the mutant strain 7+− induced by indene (final concentration 2 mM).

FIG. 19 shows indene metabolism profile of Rhodococcus sp. KY1 induced by toluene (final concentration 2 mM).

FIG. 20 shows indene metabolism profile of the mutant strain 7++ induced by toluene (final concentration 2 mM).

FIG. 21 shows indene metabolism profile of the mutant strain 7+− induced by toluene (final concentration 2 mM).

FIG. 22 shows indene metabolism profile of Rhodococcus sp. KY1 induced by naphthalene (final concentration 2 mM).

FIG. 23 shows indene metabolism profile of the mutant strain 7++ induced by naphthalene (final concentration 2 mM).

FIG. 24 shows indene metabolism profile of the mutant strain 7+− induced by naphthalene (final concentration 2 mM).

FIG. 25 shows the indene bioconversion network in Rhodococcus sp. I24 and its derivative KY1. In I24 (black arrows) and KY1 (grey arrows), different subsets of the indene bioconversion network are induced in the presence of toluene (7), naphthalene (N), or indene (1). The predominant indene oxygenation product is trans-(1R,2R)-indandiol. The naphthalene-inducible dioxygenase also catalyzes the formation of 1-indenol, as may the toluene-inducible dioxygenase. This activity, as well as a naphthalene-inducible dioxygenase activity that catalyzes the production of cis-(1R,2S)-indandiol, is present but greatly reduced in the KY-1 strain (indicated by white arrows). Toluene-grown I24 exhibits an additional dioxygenase activity, producing cis-(1S,2R)-indandiol that is absent in the KY1 strain. When indene is the sole inducer, indene oxidation proceeds mainly through a distinct, epoxide-forming monooxygenase activity to (1S,2R)-indan oxide that non-enzymatically resolves to cis-(1S,2R)- and trans-indandiol. The cis-indandiols produced in both strains are further metabolized to ketohydroxyindan.

FIG. 26 shows growth analysis of Rhodococcus sp. KY1 and the knock out strains 7++ and 7+− cultivated in medium rare containing naphthalene as sole carbon source.

FIG. 27 shows PCR primer combinations used to verify knockouts. (a) For the nimB knockout candidates, primer combinations 1 &2 and 3&4 will give a product if the pAR50 .lasmid did not integrate into the genome by homologous recomb' in. Primer pairs 1 &3 and 2&4 yield a PCR product only if the pAR50 p mid disrupts the nimB gene. (b) For the ORF5468 knockout candidates, primer combinations 1 &2 and 5&6 give a product if the pAR51 plasmid did not integrate into the genome by homologous recombination. Primer pairs 2&5 and 1 &6 yield a PCR product only if the pAR51 plasmid disrupts the ORF5486 gene.

FIG. 28 shows LB agar plates testing the ability of nimB and ORF5468 gene knockout strains to utilize naphthalene as a sole carbon source under gentamicin selection. A nimB knockouts grown on sucrose B nimB knockouts grown on naphthalene C ORF5468 knockouts grown on sucrose D ORF5468 knockouts grown on naphthalene.

### DETAILED DESCRIPTION OF THE INVENTION

1. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “Actinomycetales bacterial family” refers to the bacterial family comprised of genera, including but not limited to Actinomyces, Actinoplanes, Arcanobacterium, Corynebacterium, Dietzia, Gordonia, Mycobacterium, Nocardia, Rhodococcus, Tsukamurella, Brevibacterium, Arthrobacter, Propionibacterium, Streptomyces, Micrococcus, and Micromonospora.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. The names of the natural amino acids are abbreviated herein in accordance with the recommendations of IUPAC-IUB.

A DNA “coding sequence” is a double-stranded DNA sequence which is transcribed and translated into a polypeptide in a cell in vitro or in vivo when placed under the control of appropriate regulatory sequences. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic DNA sequences, and even synthetic DNA sequences. If the coding sequence is intended for expression in a eukaryotic cell, a polyadenylation signal and transcription termination sequence will usually be located 3′ to the coding sequence. A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

The term “complementary” is used to describe the relationship between nucleotide bases that are capable to hybridizing to one another. For example, with respect to DNA, adenosine is complementary to thymine and cytosine is complementary to guanine. Accordingly, the instant invention also includes isolated nucleic acid fragments that are complementary to the complete sequences as reported in the accompanying Sequence Listing as well as those substantially similar nucleic acid sequences.

The terms “comprise” and “comprising” is used in the inclusive, open sense, meaning that additional elements may be included.

The term “conjugative transfer” refers to the process by which one strain of bacterium transfers a plasmid to another bacterium of the same strain or of a different strain.

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

The term “disrupting or knocking out” a gene refers to removing a gene, blocking a gene's expression, or any other process which serves to decrease or remove entirely the expression of a gene.

“Donor cell” refers to a cell that may be transformed with a specified plasmid of the invention and which may conjugatively transfer that plasmid to a recipient cell. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “expression”, as used herein, refers to the transcription and stable accumulation of sense (mRNA) or antisense RNA derived from the nucleic acid fragment of the invention. Expression may also refer to translation of mRNA into a polypeptide.

A “gene” refers to an assembly of nucleotides that encode a polypeptide, and includes cDNA and genomic DNA nucleic acids. “Gene” also refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “gene product” refers to a molecule that is produced as a result of transcription of a gene. Gene products include RNA molecules transcribed from a gene, as well as proteins translated from such transcripts.

The terms “genetic region” or “coding region” will refer to a region of a nucleic acid molecule or a nucleotide sequence that comprises a gene encoding a polypeptide.

“Heterologous” DNA refers to DNA not naturally located in the cell, or in a chromosomal site of the cell. Preferably, the heterologous DNA includes a gene foreign to the cell.

As used herein, the term “homologous” in all its grammatical forms and spelling variations refers to the relationship between proteins that possess a “common evolutionary origin,” including proteins from superfamilies and homologous proteins from different species (Reeck et al., 1987, Cell 50:667). Such proteins (and their encoding genes) have sequence homology, as reflected by their high degree of sequence similarity.

The term “homologous recombination” refers to the process in which nucleic acid molecules with similar nucleotide sequences associate and exchange nucleotide strands. A nucleotide sequence of an endogenous nucleic acid which is effective for engaging in homologous recombination at a predefined position of an endogenous target nucleic acid will therefore have a nucleotide sequence which facilitates the exchange of nucleotide strands between the exogenous nucleic acid molecule and a defined position of an endogenous nucleic acid. Thus, the exogenous nucleic acid will generally have a nucleotide sequence which is sufficiently complementary to a portion of the endogenous nucleic acid molecule to promote nucleotide base pairing. Two nucleic acids must be homologous, but need not exactly correspond to each other, in order to undergo homologous recombination.

The term “including” is used herein to mean “including but not limited to”. “Including” and “including but not limited to” are used interchangeably.

The term “integration into the genome” refers to the the process by which exogenous nucleic acid molecules become succesfully incorporated within a recipient genome.

An “isolated nucleic acid molecule” or “isolated nucleic acid fragment” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester anologs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

The term “isolated polypeptide” refers to a polypeptide, which may be prepared from recombinant DNA or RNA, or be of synthetic origin, some combination thereof, or which may be a naturally-occurring polypeptide, which (1) is not associated with proteins with which it is normally associated in nature, (2) is isolated from the cell in which it normally occurs, (3) is essentially free of other proteins from the same cellular source, (4) is expressed by a cell from a different species, or (5) does not occur in nature.

A “nucleic acid” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes cDNA, genomic DNA, synthetic DNA, and semi-synthetic DNA.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein (hereinafter “Maniatis”, entirely incorporated herein by reference). The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6.times. SSC, 0.5% SDS at room temperature for 15 min, then repeated with 2.times. SSC, 0.5% SDS at 45.degree. C. for 30 min, and then repeated twice with 0.2.times. SSC, 0.5% SDS at 50.degree. C. for 30 min. A more preferred set of stringent conditions uses higher temperatures in which the washes are identical to those above except for the temperature of the final two 30 min washes in 0.2.times. SSC, 0.5% SDS was increased to 60.degree. C. Another preferred set of highly stringent conditions uses two final washes in 0.1.times. SSC, 0.1% SDS at 65.degree. C. Another set of highly stingent conditions are defined by hybridization at 0.1.times. SSC, 0.1% SDS, 65.degree. C. and washed with 2.times. SSC, 0.1% SDS followed by 0.1.times. SSC, 0.1% SDS.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Maniatis, supra, 9.50-9.51). For hybridizations with shorter nucleic acids, i.e., oligonucleotides, the position of mismatches becomes more important, and the length of the oligonucleotide determines its specificity (see Maniatis, supra, 11.7-11.8). In one embodiment the length for a hybridizable nucleic acid is at least about 10 nucleotides. Preferable a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

Suitable “nucleic acid fragments” (isolated polynucleotides of the present invention) encode polypeptides that are at least about 70% identical, preferably at least about 80% identical to the amino acid sequences reported herein. Preferred nucleic acid fragments encode amino acid sequences that are about 85% identical to the amino acid sequences reported herein. More preferred nucleic acid fragments encode amino acid sequences that are at least about 90% identical to the amino acid sequences reported herein. Most preferred are nucleic acid fragments that encode amino acid sequences that are at least about 95% identical to the amino acid sequences reported herein. Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids.

As used herein, the term “oligonucleotide” refers to a nucleic acid, generally of about 18 nucleotides, that is hybridizable to a genomic DNA molecule, a cDNA molecule, or an mRNA molecule. Oligonucleotides can be labeled, e.g., with .sup.32P-nucleotides or nucleotides to which a label, such as biotin, has been covalently conjugated. An oligonucleotide can be used as a probe to detect the presence of a nucleic acid according to the invention. Similarly, oligonucleotides (one or both of which may be labeled) can be used as PCR primers, either for cloning full length or a fragment of a nucleic acid of the invention, or to detect the presence of nucleic acids according to the invention. In a further embodiment, an oligonucleotide of the invention can form a triple helix with a DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

“Open reading frame” is abbreviated ORF and means a length of nucleic acid sequence, either DNA, cDNA or RNA, that comprises a translation start signal or initiation codon, such as an ATG or AUG, and a termination codon and can be potentially translated into a polypeptide sequence.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The terms “origin or replication” or “ORI” mean a specific site or sequence within a DNA molecule at which DNA replication is initiated.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, NY (1988); Biocomputinq: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

The term “plasmid” refers to an extra chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

“Polymerase chain reaction” is abbreviated PCR and means an in vitro method for enzymatically amplifying specific nucleic acid sequences. PCR involves a repetitive series of temperature cycles with each cycle comprising three stages: denaturation of the template nucleic acid to separate the strands of the target molecule, annealing a single stranded PCR oligonucleotide primer to the template nucleic acid, and extension of the annealed primer(s) by DNA polymerase.

The term “probe” refers to a single-stranded nucleic acid molecule that can base pair with a complementary single stranded target nucleic acid to form a double-stranded molecule.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

“Protein” (if single-chain), “polypeptide” and “peptide” are used interchangeably herein when referring to a gene product, e.g., as may be encoded by a coding sequence. When referring to “polypeptide” herein, a person of skill in the art will recognize that a protein can be used instead, unless the context clearly indicates otherwise. A “protein” may also refer to an association of one or more polypeptides. By “gene product” it is meant a molecule that is produced as a result of transcription of a gene. Gene products include RNA molecules transcribed from a gene, as well as proteins translated from such transcripts.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “recipient cell” refers to a cell which receives a plasmid via conjugative transfer from a “donor cell,” defined above.

“Regulatory region” means a nucleic acid sequence which regulates the expression of a second nucleic acid sequence. A regulatory region may include sequences which are naturally responsible for expressing a particular nucleic acid (a homologous region) or may include sequences of a different origin which are responsible for expressing different proteins or even synthetic proteins (a heterologous region). In particular, the sequences can be sequences of prokaryotic, eukaryotic, or viral genes or derived sequences which stimulate or repress transcription of a gene in a specific or non-specific manner and in an inducible or non-inducible manner. Regulatory regions include origins of replication, RNA splice sites, promoters, enhancers, transcriptional termination sequences, and signal sequences which direct the polypeptide into the secretory pathways of the target cell. A regulatory region from a “heterologous source” is a regulatory region which is not naturally associated with the expressed nucleic acid. Included among the heterologous regulatory regions are regulatory regions from a different species, regulatory regions from a different gene, hybrid regulatory sequences, and regulatory sequences which do not occur in nature, but which are designed by one having ordinary skill in the art.

The terms “restriction endonuclease” and “restriction enzyme” refer to an enzyme which binds and cuts within a specific nucleotide sequence within double stranded DNA.

The term “Rhodococcus bacterium” refers to any strain of bacterium classified within the genus Rhodococcus.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a double-stranded DNA that is complementary to and derived from mRNA. “Sense” RNA refers to RNA transcript that includes the mRNA and so can be translated into protein by the cell. “Antisense RNA” refers to a RNA transcript that is complementary to all or part of a target primary transcript or mRNA and that blocks the expression of a target gene (U.S. Pat. No. 5,107,065; WO 9928508). The complementarity of an antisense RNA may be with any part of the specific gene transcript, i.e., at the 5′ non-coding sequence, 3′ non-coding sequence, or the coding sequence. “Functional RNA” refers to antisense RNA, ribozyme RNA, or other RNA that is not translated yet has an effect on cellular processes.

The term “sequence analysis software” refers to any computer algorithm or software program that is useful for the analysis of nucleotide or amino acid sequences. “Sequence analysis software” may be commercially available or independently developed. Typical sequence analysis software will include but is not limited to the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990), and DNASTAR (DNASTAR, Inc. 1228 S. Park St. Madison, Wis. 53715 USA), and the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Publisher: Plenum, New York, N.Y.). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

The term “signal peptide” refers to an amino terminal polypeptide preceding the secreted mature protein. The signal peptide is cleaved from and is therefore not present in the mature protein. Signal peptides have the function of directing and translocating secreted proteins across cell membranes. Signal peptide is also referred to as signal protein.

A “signal sequence” is included at the beginning of the coding sequence of a protein to be expressed on the surface of a cell. This sequence encodes a signal peptide, N-terminal to the mature polypeptide, that directs the host cell to translocate the polypeptide. The term “translocation signal sequence” is used herein to refer to this sort of signal sequence. Translocation signal sequences can be found associated with a variety of proteins native to eukaryotes and prokaryotes, and are often functional in both types of organisms.

A “substantial portion” of an amino acid or nucleotide sequence comprising enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Basic Local Alignment Search Tool; Altschul, S. F., et al., (1993) J. Mol. Biol. 215:403-410; see also www.ncbi.nlm.nih.gov/BLAST/). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence. The instant specification teaches partial or complete amino acid and nucleotide sequences encoding one or more particular microbial proteins. The skilled artisan, having the benefit of the sequences as reported herein, may now use all or a substantial portion of the disclosed sequences for purposes known to those skilled in this art. Accordingly, the instant invention comprises SEQ ID NOs 1 through 6, as well as substantial portions of those sequences as defined above.

“Transcriptional and translational control sequences” are DNA regulatory sequences, such as promoters, enhancers, terminators, and the like, that provide for the expression of a coding sequence in a host cell. In eukaryotic cells, polyadenylation signals are control sequences.

A cell has been “transfected” by exogenous or heterologous DNA when such DNA has been introduced inside the cell. A cell has been “transformed” by exogenous or heterologous DNA when the transfected DNA effects a phenotypic change. The transforming DNA can be integrated (covalently linked) into chromosomal DNA making up the genome of the cell.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

A “vector” is any means for the transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. The term “vector” includes both viral and nonviral means for introducing the nucleic acid into a cell in vitro, ex vivo or in vivo. Viral vectors include retrovirus, adeno-associated virus, pox, baculovirus, vaccinia, herpes simplex, Epstein-Barr and adenovirus vectors. Non-viral vectors include plasmids, liposomes, electrically charged lipids (cytofectins), DNA-protein complexes, and biopolymers. In addition to a nucleic acid, a vector may also contain one or more regulatory regions, and/or selectable markers useful in selecting, measuring, and monitoring nucleic acid transfer results (transfer to which tissues, duration of expression, etc.). A “cloning vector” is a “replicon”, which is a unit length of DNA that replicates sequentially and which comprises an origin of replication, such as a plasmid, phage or cosmid, to which another DNA segment may be attached so as to bring about the replication of the attached segment. Cloning vectors may be capable of replication in one cell type, and expression in another (“shuttle vector”).

The term “selectable marker” means an identifying factor, usually an antibiotic or chemical resistance gene, that is able to be selected for based upon the marker gene's effect, i.e., resistance to an antibiotic, wherein the effect is used to track the inheritance of a nucleic acid of interest and/or to identify a cell or organism that has inherited the nucleic acid of interest.

The following abbreviations are used throughout the specification. The term “AmpR” as used herein refers to ampicillin resistance. The term “CFU” as used herein refers to a colony forming unit. The term “GntR” as used herein refers to gentamicin resistance. The term “KanR” as used herein refers to kanamycin resistant. The term “ORF” as used herein refers to an open reading frame. The term “PFGE” as used herein refers to pulsed field gel electrophoresis. The term “RifR” as used herein refers to rifampicin resistant. The term “StrR” as used herein refers to streptomycin resistant. The term “TsrR” as used herein refers to thiostrepton resistant.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

These embodiments of the present invention, other embodiments, and their features and characteristics will be apparent from the description, drawings, and claims that follow.

2. General

We isolated and sequenced pB264, a 4,970 bp cryptic plasmid from Rhodococcus sp. B264-1 with features of a theta-type replication mechanism. Derivatives of pB264 replicate in a diverse range of Rhodococcus species, showing that this plasmid does not bear the same host range restrictions that have been exhibited by other theta replicating plasmids. Replication or maintenance of pB264 is inhibited at 37° C., making pB264 useful as a suicide vector for genetic manipulation of Rhodococcus. A series of deletions revealed that ca. 1.3 kb from pB264 was sufficient to support replication and stable inheritance of the plasmid. This region includes two open reading frames that encode functions (RepAB) that can support replication of pB264 derivatives in trans. Rhodococcus sp. B264-1 will mobilize pB264 into other Rhodococcus species via conjugation, making it possible to genetically modify bacterial strains that are otherwise difficult to transform. The cis-acting element (oriT) required for conjugal transfer of pB264 resides within a ca. 0.7 kb region that is distinct from the regions responsible for replication.

Shuttle vectors derived from pB264 will be useful for genetic studies and strain improvement in Rhodococcus. In particular (by taking advantage of the transmissibility of pB264), this plasmid will be useful in cases where the target strain of Rhodococcus is difficult to transform directly. The plasmid is also useful for introducing or expressing heterologous or homologous genes in Rhodococcus strains. The plasmid is also useful for generating chromosomal insertions into Rhodococcus. From a research point of view, pB264 will also be useful for studying the processes of theta replication and conjugal transfer among actinomycetes.

Indeed, using a targeted gene knockout technique, we were able to inactivate the nimA, nimB and ORF5468 genes in KY1. The nimA strain lost the ability to convert indene into cis- and/or trans-indandiol, which indicates that the nimA gene encodes at least a subunit of the monooxygenase enzyme. Both the nimB and the ORF5468 gene knockout strains lost the ability to grow on naphthalene, suggesting that the genes play a critical role in naphthalene metabolism.

3. Nucleic Acids, Plasmids, and Transformed Host Cells

The present invention provides novel nucleic acids and plasmids comprising such nucleic acids for the manipulation of genes in Rhodococcus and other members of the Actinomycetales bacterial family.

In certain embodiments, the isolated nucleic acids sequences of the invention support replication in Rhodococcus and may comprise a nucleic acid sequence selected from the group consisting of: (a) SEQ ID NO: 2; (b) a nucleic acid sequence that hybridizes with (a) under stringent hybridization conditions; and (c) a nucleic acid sequence that is complementary to (a) or (b). In other embodiments, such isolated nucleic acids may comprise a nucleic acid sequence selected from the group consisting of: (a) SEQ ID NO: 5; (b) SEQ ID NO: 6; (c) SEQ ID NO: 4; (d) a sequence that hybridizes with (a), (b), or (c) under stringent hybridization conditions; and (e) a nucleic acid sequence that is complementary to (a), (b), (c), or (d). In still other embodiments, such nucleic acids may comprise a nucleic acid sequence selected from the group consisting of: (a) a nucleic acid sequence comprising SEQ ID NOs: 4, 5, and 6; (b) a nucleic acid sequence comprising SEQ ID NOs: 5 and 6; (c) a nucleic acid sequence comprising SEQ ID NOs: 4 and 5; (d) a nucleic acid sequence comprising SEQ ID NOs: 4 and 6; (e) a nucleic acid sequence that hybridizes with (a), (b), (c) or (d) under stringent hybridization conditions; and (f) a nucleic acid sequence that is complementary to (a), (b), (c), (d), or (e). In certain embodiments, the isolated nucleic acids may support conditional replication, i.e. replication that is temperature sensitive.

In other embodiments, the isolated nucleic acids of the invention are necessary for conjugative transfer of a plasmid in one Rhodococcus bacterium to another bacterium. In certain embodiments, such isolated nucleic acids may comprise a nucleic acid sequence selected from the group consisting of: (a) SEQ ID NO: 3; (b) a nucleic acid sequence that hybridizes with (a) under stringent hybridization conditions; and (c) an isolated nucleic acid that is complementary to (a) or (b). In other embodiments, such isolated nucleic acids may comprise a nucleic acid sequence selected from the group consisting of: (a) the origin of Transfer (oriT) sequence; (b) a nucleic acid sequence that hybridizes with (a) under stringent hybridization conditions; and (c) a nucleic acid sequence that is complementary to (a) or (b). In certain embodiments, a nucleic acid sequence that enables conjugative transfer of the plasmid may comprise a nucleic acid selected from the group consisting of: (a) a nucleic acid sequence comprising the region involved in conjugal transfer containing origin of Transfer (oriT) sequence; (b) a nucleic acid sequence comprising the origin of Transfer (oriT) sequence; (c) a nucleic acid sequence that hybridizes with (a) or (b) under stringent hybridization conditions; and (d) a nucleic acid sequence that is complementary to (a), (b) or (c).

The present invention also provides methods for identifying plasmids with properties of temperature sensitivity in Rhodococcus. For example, such a method may comprise: (a) isolating DNA from a Rhodococcus bacterium that cannot grow well above a minimum temperature; (b) examining the DNA for the presence of small plasmids; (c) extracting the small plasmids; (d) testing the small plasmids for temperature sensitive replication in Rhodococcus strains that grow above the minimum temperature. Further, the present invention also provides methods for identifying plasmids with the ability to conjugatively transfer from one Rhodococcus bacterium to another bacterium. For example, such methods may comprise: (a) isolating DNA from a Rhodococcus bacterium; (b) examining the DNA for the presence of small plasmids; (c) extracting the small plasmids; (d) transforming a Rhodococcus bacterium with the small plasmid; (e) culturing the transformed Rhodococcus bacterium of (d) for a length of time and under conditions with another bacterium, such that conjugative transfer is facilitated, wherein if conjugative transfer is observed, the small plasmid has the ability to conjugatively transfer.

Bacterial plasmids typically range in size from about 1 kb to about 200 kb and are generally autonomously replicating genetic units in the bacterial host. When a bacterial host has been identified that may contain a plasmid containing desirable genes, cultures of host cells are growth up, lysed and the plasmid purified from the cellular material. If the plasmid is of the high copy number variety, it is possible to purify it without additional amplification. If additional plasmid DNA is needed, a bacterial cell may be grown in the presence of a protein synthesis inhibitor such as chloramphenical which inhibits host cell protein synthesis and allow additional copies of the plasmid to be made. Cell lysis may be accomplished either enzymatically (i.e lysozyme) in the presence of a mild detergent, by boiling or treatment with strong base. The method chosen will depend on a number of factors including the characteristics of the host bacteria and the size of the plasmid to be isolated. After lysis the plasmid DNA may be purified by gradient centrifugation (CsCl-ethidium bromide for example) or by phenol:chloroform solvent extraction. Additionally, size or ion exchange chromatography may be used as well as differential separation with polyethylene glycol. Once the plasmid DNA has been purified, the plasmid may be analyzed by restriction enzyme analysis and sequenced to determine the sequence of the genes contained on the plasmid and the position of each restriction site to create a plasmid restriction map. Methods of constructing or isolating vectors are common and well known in the art (see for example Maniatis supra, Chapter 1; Rohde, C., World J. Microbiol. Biotechnol. (1995), 11(3), 367-9); Trevors, J. T., J. Microbiol. Methods (1985), 3(5-6), 259-71).

The present invention also provides plasmids comprising at least one of the above described isolated nucleic acids. In certain embodiments, the plasmids of the invention comprise a nucleic acid sequence that supports replication and a nucleic acid sequence that is necessary for conjugative transfer of a plasmid in one Rhodococcus bacterium to another bacterium. In certain embodiments, the replication of the plasmid is temperature-sensitive. For example, in certain plasmids, the ability to replicate of the plasmid is reduced at temperatures above 30° C. In certain embodiments, the plasmid may comprise SEQ ID NO: 1. Such plasmid may enable conditional replication of the plasmid in a donor cell and is able to be transmitted via conjugative transfer to another cell.

The subject plasmids may be modified for gene expression in a Rhodococcus strain. For example, the plamid may further comprise gene constructs comprising at least one promoter suitable for the expression of a gene in a Rhodococcus strain and a nucleic acid sequence encoding a gene to be expressed. The present invention also provides methods of using such plasmids in a method for the expression of a nucleic acid in a Rhodococcus bacterium. For example, such a method may comprise: a) providing a plasmid of the invention modified for the expression of a gene; b) transforming a Rhodococcus bacterium with the plasmid of (a); and c) culturing the transformed Rhodococcus bacterium of (b) for a length of time and under conditions whereby the nucleic acid sequence encoding a gene to be expressed is expressed.

Plasmids useful for gene expression in bacteria may be either self-replicating (autonomously replicating) plasmids or chromosomally (e.g. genomically) integrated. The self-replicating plasmids have the advantage of having multiple copies of genes of interest, and therefore the expression level can be very high. Chromosome integration plasmids are integrated into the genome by recombination and are thus stably integrated.

The plasmids or vectors according to the invention may further comprise at least one promoter suitable for driving expression of a gene in Rhodococcus. Typically these promoters including the initiation control regions will be derived from a Rhodococcus sp. Termination control regions may also be derived from various genes native to the preferred hosts. Optionally, a termination site may be unnecessary, however, it is most preferred if included.

Vectors or plasmids useful for the transformation of suitable host cells are well known in the art. Typically the vector or plasmid contains sequences directing transcription and translation of the relevant gene, a selectable marker, and sequences allowing autonomous replication or chromosomal integration. In a specific embodiment, the plasmid or vector comprises a nucleic acid according to the present invention. Suitable vectors comprise a region 5′ of the gene which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host. Vectors of the present invention may additionally contain a sequence as described above that facilitates the replication of the vector in the Rhodococcus host. The vectors of the present invention may contain convenient restriction sites for the facile insertion of genes of interest to be expressed in the Rhodococcus host.

The present invention also relates to a plasmid or vector that is able to replicate or “shuttle” between at least two different organisms. Shuttle vectors are useful for carrying genetic material from one organism to another. The shuttle vector is distinguished from other vectors by its ability to replicate in more than one host. This is facilitated by the presence of an origin of replication corresponding to each host in which it must replicate. The present vectors are designed to replicate in Rhodococcus for the purpose of gene expression and manipulation.

Many of the genetic manipulations for this vector may be easily accomplished in E. coli. It is therefore particularly useful to have a shuttle vector comprising an origin of replication that will function in E. coli and other gram positive bacteria. Accordingly, the plasmids of the invention may additionally comprise such origin of replication. A number of ORI sequences for gram positive bacteria have been determined and the sequence for the ORI in E. coli determined (see for example Hirota et al., Prog. Nucleic Acid Res. Mol. Biol. (1981), 26, 33-48); Zyskind, J. W.; Smith, D. W., Proc. Natl. Acad. Sci. U.S.A., 77, 2460-2464 (1980), GenBank ACC. NO. (GBN): J01808). For example, ORI sequence that may be used in the present invention include those ORI sequences isolated from gram positive bacteria, and particularly those members of the Actinomycetales bacterial family.

The present invention further provides a transformed bacterium comprising any of the above-described plasmids. Such bacterium may be of the genus Rhodococcus, for example, one selected from the group including, but not limited to, Rhodococcus equi, Rhodococcus erythropolis, Rhodococcus opacus, Rhodococcus rhodochrous, Rhodococcus globerulus, Rhodococcus koreensis, Rhodococcus fascians, Rhodococcus I24, Rhodococcus KY1, Rhodococcus B264-1, and Rhodococcus ruber. However, any bacterium of the Actinomycetales bacterial family may comprise a plasmid of the present invention.

Introduction of the plasmid into the host bacterium may be accomplished by known procedures such as by transformation, e.g., using calcium-permeabilized cells, electroporation, transduction, or by transfection using a recombinant phage virus. (Maniatis, supra) The present vectors may be co-transformed with additional vectors, also containing DNA heterologus to the host. It will be appreciated that both the present vector and the additional vector will have to reside in the same incompatibility group. The ability for two or plasmids to coexist in same host will depend on whether they belong to the same incompatibility group. Generally, plasmids that do not compete for the same metabolic elements will be compatible in the same host. For a compete review of the issues surrounding plasmid coexistence see Thomas et al., Annu. Rev. Microbiol. (1987), 41, 77-101.

Materials and methods suitable for the maintenance and growth of bacterial cultures are well known in the art. See, e.g. Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989).

4. Methods of Use

The present invention also provides methods for controlling replication of a temperature sensitive plasmid in a Rhodococcus bacterium. For example, such a method may comprise: a) providing a plasmid that is unable to replicate above a certain temperature; b) transforming a Rhodococcus bacterium with the plasmid of (a); and c) controlling the replication of the plasmid by adjusting the temperature of the culture. In certain embodiments, the replication is inhibited by elevating the temperature.

The present invention also provides methods for effecting conjugative transfer of plasmids between Rhodococcus strains. For example, such a method may comprise: a) providing a plasmid with the ability to conjugatively transfer from one Rhodococcus bacterium to another bacterium; b) transforming a Rhodococcus bacterium with the plasmid of (a); and c) culturing the transformed Rhodococcus bacterium of (b) for a length of time and under conditions with another Rhodococcus bacterium such that conjugative transfer occurs.

Still further, the present invention provides methods for selecting from a culture a recipient Rhodococcus bacterium that has integrated a plasmid into its genome comprising the use of temperature selection. For example, the recipient strain may have integrated the plasmid via homologous recombination, or may have integrated the plasmid through another means of delivering DNA into the genome as known in the art, for example, transposition of a DNA element from the plasmid into the genome. In such methods, temperature selection permits distinction or selection of those bacteria that have integrated a plasmid or portion thereof into the genome. In certain embodiments, such methods may comprise conjugatively transferring from a donor Rhodococcus bacterium to a recipient Rhodococcus bacterium that can grow above a minimum temperature a plasmid that cannot replicate above the minimum temperature; elevating the temperature of the culture above the minimum temperature for a sufficient time to prevent replication of the plasmid and survival of the donor Rhodococcus bacterium, whereby only a recipient Rhodococcus bacterium that has incorporated the plasmid into its genome survives such temperature elevation. In certain embodiments of these methods, the plasmid may comprises (a) the minimal replicon sequence; (b) a sequence that hybridizes with (a) under stringent hybridization conditions; and (c) a sequence that is complementary to (a) or (b). In some embodiments, the minimum temperature in 30° C. and the temperature is elevated to 37° C. In some embodiments, the donor Rhodococcus bacterium is of the strain Rhodococcus B264-1 and the recipient Rhodococcus bacterium is of the strain Rhodococcus I24.

An alternative method for introducing a subject plasmid into the recipient strain and selecting for homologous recombination of the element in the recipient strain comprises the following. The plasmid for use in such a method comprises a region of homology (to drive homologous replication with a gene/DNA in the recipient strain), the cis-acting origin of transfer (oriT, or the region defined previously as the region responsible for conjugal trasnfer of the plasmid (SEQ ID NO: 3) and the cis-acting component of the origin of replication (oriV, SEQ ID NO: 4). However, the plasmid lacks one or more of the trans-acting components of replcation (e.g. repA or repB, SEQ ID NOs: 5 and 6) which are supplied via a separately replicating element in the donor strain, e.g. a strain such as Rhodococcus sp. B264-1 expresses repA and/or repB from the chromosome or from one plasmid, and expression of this/these genes permits replication the other plasmid carrying the cis-acting origin of replication (oriV) derived from pB264. Thus, replication of the pB264 derivative is completely dependent on factors provided only in the donor strain. Once this plasmid is tranferred to the recipient strain via conjugation, it will be unable to replicate autonomously because of the lack of repA and/or repB in the recipient strain. Thus the plasmid would be rapidly lost from the recipient cells if it were not stabilized somehow. Homologous recombination, through which the plasmid integrates into the genome of the recipient strain, is one of the more likely scenarios for stabilizing the plasmid. Thus, when selective pressure is applied to identify recipients that have retained the plasmid (or selectable markers such as antibiotic resistance markers that are associated with the plasmid), cells that have integrated the incoming plasmid into their genome will be more likely to survive. An advantage of this embodiment is that it can be used with recipient strains for which higher temperature growth cannot be used as a means of eliminating the donor strain following conjugation. That is, rather than exploiting temperature-senstitive replication of the transferred plasmid to identify integration into the genome, we would be exploiting the conditional replication of the plasmid only in the donor strain. In this scenario, a separate means for eliminating the donor strain would also have to be employed (e.g. differential sensitivity of the donor and recipient strains to an antibiotic other than one used to select the plasmid directly.)

Still further, the present invention provides methods for manipulating genes in Rhodococcus and other bacteria. For example, in certain embodiments, a method for introducing a nucleic acid into a Rhodococcus bacterium may comprise culturing a first strain of Rhodococcus bacterium transformed with a plasmid comprising a nucleic acid sequence that enables conjugative transfer of the plasmid with a second strain of Rhodococcus bacterium for a length of time under conditions such that conjugative transfer occurs between the first and second strains of bacterium, whereby the plasmid is transferred from the first strain of bacterium to the second strain of bacterium. In certain embodiments, the plasmid may further comprise a gene construct, for example, encoding a protein to be expressed in the second strain of bacterium. Such gene construct may further comprise at least one promoter suitable for the expression of a gene in a Rhodococcus bacterium. The gene may be expressed in the second strain of Rhodococcus bacterium following the transfer. In certain embodiments, the plasmid may comprise a mutant gene or a fragment of a gene, e.g. to use in gene disruption or knock-out. In certain embodiments, the plasmid or a portion thereof may be integrated into the genome of the second strain of bacterium, for example, by homologous recombination. In certain embodiments, the replication of the plasmid may be temperature sensitive, and the plasmid may comprise (a) SEQ ID NO: 2; (b) a sequence that hybridizes with (a) under stringent hybridization conditions; and (c) a sequence that is complementary to (a) or (b). In methods wherein the plasmid replication is temperature sensitive, the method may further comprise the step of selecting a bacterium of the second strain that has integrated the plasmid or a portion thereof into its genome by elevating the temperature of the culture.

Endogenous genes of interest for expression in a Rhodococcus using the subject plasmids and methods include, but are not limited to: a) genes encoding enzymes involved in the production of isoprenoid molecules, for example, 1-deoxyxylulose-5-phosphate synthase gene (dxs) can be expressed in Rhodococcus to exploit the high flux for the isoprenoid pathway in this organism; b) genes encoding polyhydroxyalkanoic acid (PHA) synthases (phaC) which can also be expressed for the production of biodegradable plastics; c) genes encoding carotenoid pathway genes (eg, crtl) can be expressed to increase pigment production in Rhodococcus; d) genes encoding nitrile hydratases for production of acrylamide in Rhodococcus and the like, and d) genes encoding monooxygenases derived from waste stream bacteria.

Heterologous genes of interest for expression in a Rhodococcus include, but are not limited to: a) ethylene forming enzyme (efe) from Pseudomonas syringae for ethylene production, b) pyruvate decarboxylase (pdc), alcohol dehydrogenase (adh) for alcohol production, c) terpene synthases from plants for production of terpenes in Rhodococcus, d) cholesterol oxidase (choD) from Mycobacterium tuberculosis for production of the enzyme in Rhodococcus; and the like, and e) genes encoding monooxygenases derived from waste stream bacteria.

Optionally it may be desired to produce the instant gene product as a secretion product of the transformed host. Secretion of desired proteins into the growth media has the advantages of simplified and less costly purification procedures. It is well known in the art that secretion signal sequences are often useful in facilitating the active transport of expressible proteins across cell membranes. The creation of a transformed host capable of secretion may be accomplished by the incorporation of a DNA sequence that codes for a secretion signal which is functional in the host production host. Methods for choosing appropriate signal sequences are well known in the art (see for example EP 546049; WO 9324631). The secretion signal DNA or facilitator may be located between the expression-controlling DNA and the instant gene or gene fragment, and in the same reading frame with the latter.

Once a suitable Rhodococcus host is successfully transformed with the appropriate vector of the present invention it may be cultured in a variety of ways to allow for the commercial production of the desired gene product. For example, large scale production of a specific gene product, overexpressed from a recombinant microbial host may be produced by both batch or continuous culture methodologies.

A classical batch culturing method is a closed system where the composition of the media is set at the beginning of the culture and not subject to artificial alterations during the culturing process. Thus, at the beginning of the culturing process the media is inoculated with the desired organism or organisms and growth or metabolic activity is permitted to occur adding nothing to the system. Typically, however, a “batch” culture is batch with respect to the addition of carbon source and attempts are often made at controlling factors such as pH and oxygen concentration. In batch systems the metabolite and biomass compositions of the system change constantly up to the time the culture is terminated. Within batch cultures cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the Fed-Batch system. Fed-Batch culture processes are also suitable in the present invention and comprise a typical batch system with the exception that the substrate is added in increments as the culture progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO.sub.2. Batch and Fed-Batch culturing methods are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36, 227, (1992), herein incorporated by reference.

Commercial production of the instant proteins may also be accomplished with a continuous culture. Continuous cultures are an open system where a defined culture media is added continuously to a bioreactor and an equal amount of conditioned media is removed simultaneously for processing. Continuous cultures generally maintain the cells at a constant high liquid phase density where cells are primarily in log phase growth. Alternatively continuous culture may be practiced with immobilized cells where carbon and nutrients are continuously added, and valuable products, by-products or waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the culture. Methods of modulating nutrients and growth factors for continuous culture processes as well as techniques for maximizing the rate of product formation are well known in the art of industrial microbiology and a variety of methods are detailed by Brock, supra.

In one embodiment, the present invention provides a method for disrupting or knocking out a gene in a Rhodococcus bacterium, comprising culturing a first strain of Rhodococcus bacterium transformed with a plasmid comprising a nucleic acid sequence that enables conjugative transfer of the plasmid and a nucleic acid sequence that serves to disrupt or knock out the gene with a second strain of Rhodococcus bacterium for a length of time under conditions such that conjugative transfer occurs between the first and second strains of bacterium, whereby the plasmid is transferred from the first strain of bacterium to the second strain of bacterium and the sequence that serves to disrupt or knock out the gene is integrated into the genome of the second second strain of bacterium. Such integration may occur by homologous recombination. For example, a sequence that is homologous to a gene or other DNA element in the second strain can serve as a substrate for homologous recombination, and thereby disrupting the gene or DNA element via insertion.

A “nucleic acid sequence that serves to disrupt or knock out a gene” in certain embodiments refers to a uniquely configured fragment of nucleic acid which is introduced into a recipient cell and allowed to recombine with the genome at the chromosomal locus of the gene of interest to be mutated. Thus a given knock out construct is specific for a given gene to be targeted for disruption. Nonetheless, many common elements exist among these constructs and these elements are well known in the art. A typical knock out construct contains nucleic acid fragments of not less than about 0.5 kb nor more than about 10.0 kb from both the 5′ and the 3′ ends of the genomic locus which encodes the gene to be mutated. These two fragments are separated by an intervening fragment of nucleic acid which encodes a positive selectable marker, such as the neomycin resistance gene (neo^{R}). The resulting nucleic acid fragment, consisting of a nucleic acid from the extreme 5′ end of the genomic locus linked to a nucleic acid encoding a positive selectable marker which is in turn linked to a nucleic acid from the extreme 3′ end of the genomic locus of interest, omits most of the coding sequence for the gene of interest to be knocked out. When the resulting construct recombines homologously with the chromosome at this locus, it results in the loss of the omitted coding sequence, otherwise known as the structural gene, from the genomic locus. A recipient cell in which such a rare homologous recombination event has taken place can be selected for by virtue of the stable integration into the genome of the nucleic acid of the gene. Variations on this basic technique also exist and are well known in the art.

Other methods for disrupting or knocking out a gene, such as transpostion of a DNA element from the plasmid into the genome, are well-known in the art and within the scope of the present invention.

5. Kits

The present invention also provides kits for gene expression and manipulation in Rhodococcus and other bacteria. In certain embodiments, the kit comprises an isolated nucleic acid of the invention. In certain embodiments, the nucleic acid comprises a plasmid of the invention. In yet other embodiments, the kit may comprise a culture of transformed bacteria of the present invention. Kit components may be packaged for either manual or partially or wholly automated practice of the foregoing methods. In other embodiments involving kits, instructions for their use may be provided.

### EXEMPLIFICATION

The present invention now being generally described, it may be more readily understood by reference to the following examples which are included merely for purposes of illustration of certain aspects and embodiments of the present invention, and are not intended to limit the invention.

### Example 1

### Exemplary General Methods for Practice

General DNA Manipulation

Restriction enzymes, Klenow fragment of DNA polymerase and molecular weight markers were purchased from New England Biolabs (Beverly, Mass.) and used as specified by the manufacturer. The pCR-Script cloning kit was purchased from Stratagene (La Jolla, Calif.) and the GeneClean II kit was purchased from Bio101 (Vista, Calif.), and both were used according to the manufacturers' guidelines. DNA sequencing of pB264 was carried out initially by Lark Technologies (Houston, Tex.), while ambiguities were resolved and additional sequencing was carried out at the MIT Biopolymers Lab using Applied Biosystems BigDye Terminator cycle sequencing reagents (Applied Biosystems, Foster City, Calif.). Large scale plasmid purification from E. coli cultures were carried out using the Wizard Plus Maxiprep kit (Promega, Madison, Wis.).

Strains and Plasmids

Principal strains and plasmids used in this invention are described in Table 1.

TABLE 1
Key plasmids and strains used in this invention.

| Bacterial Strains | Description | Source or Reference |
|---|---|---|
| Escherichia coli XL1-Blue | used for routine plasmid manipulation | Stratagene, La Jolla, CA |
| Rhodococcus sp. B264-1 | Source of pB264; cannot grow above 32° C. | [13] |
| Rhodococcus sp. B264-1 R1 | spontaneous RifR-derivative of B264-1 | this invention |
| Rhodococcus sp. I24 | orange colonies; grows well at 37° C. | [13] |
| Rhodococcus sp. I24 R6 | RifR-derivative of I24 | this invention |
| R. erythropolis SQ1 | buff-colored colonies; RifR, StrR; | [8] |
| | derivative of ATCC4277 | |
| Rhodococcus KY1 | derivative of Rhodococcus sp. I24, lacking | Yanagimachi et al., 2001 |
| | the 340 kb megaplasmid; naph⁺, tol^{-(a)} | |
| 50A2 | Rhodococcus KY1 mutant with nimB | this invention |
| | knocked out; GentR | |
| 50B3 | Rhodococcus KY1 mutant with nimB | this invention |
| | knocked out; GentR | |
| KY1 (pAR51)a | Rhodococcus KY1 mutant with ORF5468 | this invention |
| | kcocked out; GentR | |
| KY1 (pAR51)b | Rhodococcus KY1 mutant with ORF5468 | this invention |
| | knocked out; GentR | |
| Plasmids | | |
| pAR50 | Small internal fragment of nimB from | this invention |
| | Rhodococcus I24 ligated into pAL298, | |
| | GentR | |
| pAR51 | Small internal fragment of ORF5468 | this invention |
| | from Rhodococcus I24 ligated into | |
| | pAL298, GentR | |
| pAL220 | pB264, ligated into pCR-Script | this invention |
| pAL224 | KanR marker from pUC4K ligated as an | this invention |
| | EcoRI fragment into the EcoRI site of | |
| | pAL220 | |
| pAL231 | TsrR marker from pGM160 ligated as an | this invention |
| | EcoRI-SmaI fragment into the EcoRI and | |
| | EcoRV sites of pAL220 | |
| pAL281 | GntR plasmid carrying NG2 origin for | this invention |
| | replication in Rhodococcus and E. coli | |
| pAL282 | Replicates at 37° C. in Rhodococcus and | this invention |
| | E. coli, GentR | |
| pAL298 | GntR marker from pGM160 ligated as an | this invention |
| | EagI fragment into the NotI site (partial | |
| | digest) of pAL220 | |
| pAL302 | Amp^{r}, aacC1, ColE1, contains internal part | this invention |
| | of nimA (nimA*), replicates in both E. coli | |
| | and Rhodococcus | |
| pAL305 | PstI deletion derivative of pAL298 lacking | this invention |
| | majority of pB264 element | |
| pAL312 | ORF7 from pB264 positioned downstream | this invention |
| | of constitutive trc promoter in a plasmid | |
| | bearing the NG2 origin and a spectinomycin | |
| | resistance marker | |
| pAL314 | ORF7 and ORF8 from pB264 positioned | this invention |
| | downstream of constitutive trc promoter in | |
| | a plasmid bearing the NG2 origin and a | |
| | spectinomycin resistance marker | |
| pB264 | 4970 bp cryptic plasmid from Rhodococcus | this invention |
| | sp. B264-1 | |
| pCR-Script | AmpR plasmid for cloning blunt-end DNAs | Stratagene, La Jolla, CA |
| pGM160 | source of GntR and TsrR markers | [36] |
| pJANET | majority of pB264 in vector carrying NG2 | this invention |
| | origin of replication and GntR | |
| pUC4K | source of KanR | Amersham Pharmacia, |
| | | Piscataway, NJ |
| pXS9a | KanR marker from pUC4K ligated as an | this invention |
| | EcoRI fragment into the EcoRI site of | |
| | pAL231 | |

^{(a)}Abbreviations: napt^{+/-}, ability/inability to use naphthalene as a carbon source; tol^{+/-}, ability/inability to use toluene as a carbon source

Selection of rifampicin resistant strains. The B264-1 R1 strain of Rhodococcus was isolated by plating a culture of Rhodococcus sp. B264-1 that had been grown in Luria-Bertani (LB) medium; onto LB 2% agar supplemented with 20 mg/l rifampicin. One of the RifR colonies that arose was designated B264-1 RI, and was found to be morphologically indistinguishable from the wild-type strain. Rhodococcus sp. I24 R6 was generated by treating an I24 culture with ethylmethanesulfonate by the method of Eisenstadt et al and selecting for growth on LB 2% agar supplemented with 20 mg/l rifampicin. One of the resulting colonies, all of which were morphologically indistinguishable from the wild-type I24 strain, was designated I24 R6.

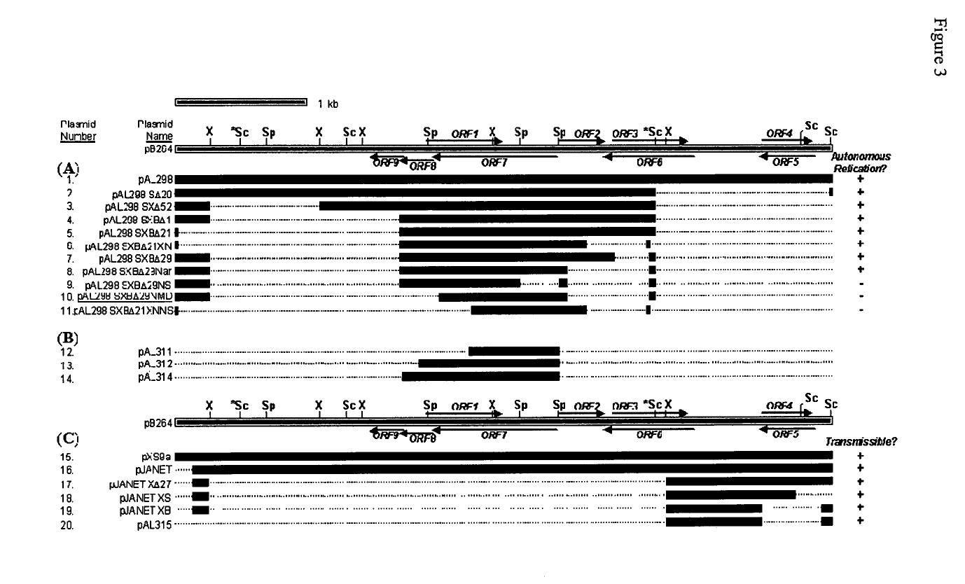

Construction of deletion series describe in FIG. 3. Deletions were carried out as follows: Partial digestion of plasmid 1 with SacII followed by religation of the products generated plasmid 2. Partial digest of plasmid 2 with XhoI produced plasmid 3. Partial digest of plasmid 3 with BsaHI produced plasmid 4. Plasmids 5 and 7 were both derived from partial digest of plasmid 4 with BsaHI. Plasmid 6 was prepared by complete digestion of plasmid 5 with XmnI and partial digestion with NaeI. Deleting the NarI fragment from plasmid 7 produced plasmid 8. Partial digestion of plasmid 8 with SphI generated plasmid 9. Deleting the region from an MfeI site in the pB264 element to a DraI site within the ColEI origin of the vector of plasmid 9 generated plasmid 10, whereas deleting a region from the NaeI site within the pB264 element to a ScaI site within the vector of plasmid 6 produced plasmid 11. Termini for these deletions were confirmed by DNA sequencing. Plasmids 12 and 13 (pAL311 and pAL312) were prepared as described below. Plasmid 14 (pAL314) was prepared by ligating an AatII-PvuII fragment from plasmid 8 into the AatII and SpeI(blunted) sites of plasmid 12, which positioned both ORF7 and ORF8 downstream of the trc promoter. Plasmid 17 was prepared by a partial XhoI deletion of plasmid 15. Plasmids 18 and 19 were created by SmaI and partial BsaHI deletion of plasmid 17, respectively. Plasmid 20 (pAL315) was prepared by ligating the XhoI-PstI fragment from plasmid 19 into the XhoI and PstI sites of pAL281.

Construction of pAL311 and pAL312. The plasmid pJP10 was obtained from J. Parker (MIT Dept. of Biology). This plasmid contains the NG2 origin of replication from pEP2, the RP4 mob element from pSUP301, a spectinomycin resistance marker derived from the omega interposon, a KanR marker from pUC4K, and lacl^{q} and the trc promoter from pTrc99A (Amersham Pharmacia, Piscataway, N.J.). pJP10 also possesses a useful polylinker positioned downstream of the trc promoter. Deleting the KanR marker and the lacl^{q} gene as a PvuII fragment from pJP10 produced pAL307. A 0.7 kb portion of ORF7 was amplified from pB264 by polymerase chain reaction using the primers ORF7 3′ (5′-GGAATTCGCTGTCATGAGTGGGCAGGT-3′) and ORF7 5′ (5′-CCTGCAGAAATGCCCAGGTTTCGTTCGAGA-3′) and ligated into pCR2.1-TOPO using the TOPO-TA Cloning Kit (Invitrogen, Carlsbad, Calif.), creating the plasmid pAL308. The ORF7 fragment was then positioned downstream of the trc promoter by ligating the PstI-SpeI fragment from pAL308 into the PstI and SpeI sites of pAL307, to make the plasmid pAL311. pAL312 was prepared by ligating an AatII-SmaI fragment from pAL298 SXBΔ29Nar (plasmid 8, FIG. 3) into the AatII and SpeI (blunted with the Klenow fragment of DNA polymerase I) sites of pAL311, which reconstitutes ORF7.

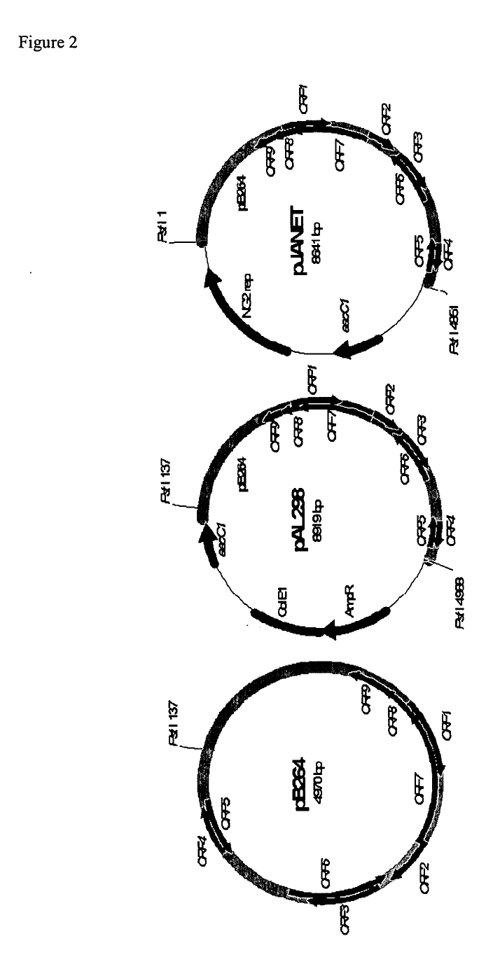

Construction of pJANET The plasmid pAL280 carries the NG2 origin of replication and a GntR marker, as well as polylinker positioned upstream of a promoterless β-glucuronidase gene from E. coli (Priefert et al., submitted). The majority of the β-glucuronidase gene was deleted from this plasmid as a SnaBI-XmnI fragment, leaving pAL281, which bore the polylinker, the NG2 origin and the GntR marker. pJANET (FIG. 2) was created by ligating the majority of pB264 as a PstI fragment from pAL220 into the PstI site of pAL281.

Transformation

Direct transformation of bacterial strains was carried out via electroporation. Electroporation of E. coli followed the protocol described in the BioRad (Richmond, Calif.) Gene Pulser Manual. Electroporation of R. erythropolis SQ1 followed the protocol of Treadway et al. A protocol developed for electroporation of Corynebacterium was used for transformation of Rhodococcus sp. B264-1. Rhodococcus sp. I24 and KY1 was prepared for electroporation as follows. Approximately 0.2-0.5 ml of a late exponential stage culture was added to 200 ml MB 1.5% glycine (per liter, 5 g yeast exract, 15 g BactoTryptone, 5 g BactoSoytone, 5 g NaCl, 15 g glycine) for I24 cells or TBS glycine (17 g bacto tryptone; 3 g bacto soytone; 5 g NaCl; 2.5 g dipotassium phosphate; 5 g yeast extract; 15 g glycine) for KY1 cells, and incubated with aeration at 37° C. until O.D.₆₀₀ was approximately 0.25 (10-14 hours); cells from this culture were centrifuged and washed twice with 30 ml ice cold EPB1 (20 mM Hepes, 5% glycerol, pH7.2) and once with 10 ml ice cold EPB2 (5 mM Hepes, 15% glycerol, pH7.2). The final cell pellet was resuspended in ca. 1 ml of EPB2 and aliquots were stored at −80° C. 0.5-10 μg DNA was mixed with 70 μl cells and electroporated in 2 mm cuvettes with a BioRad Gene Pulser (400 Ω, 25 μFd, 2.5 kV). Cells recovered for a minimum of one hour at a suitable temperature (30-37° C., depending on the plasmid DNA involved) and were plated onto LB 2% agar with appropriate antibiotics. Following transformation of Rhodococcus cells, putative transformants were routinely tested by isolating total genomic DNA (see below), and carrying out diagnostic restriction enzyme digests. In cases where plasmids could not be easily observed on agarose gels after restriction enzyme digest (as was usually the case with Rhodococcus sp. I24 transformants), Southern blots or, more routinely, a “retransformation” strategy was employed to detect the plasmids (see below).

Preparation of Genomic DNA from Rhodococcus Cells

Cells from 7 ml of late exponential culture were centrifuged, the supernatant discarded, and the cell pellet frozen (for no longer than one hour at −20° C.; if pellets were to be kept for longer, they were stored at −80° C.). Cells were then thawed and resuspended in 250 μl fresh 10 mg/ml lysozyme in TE (10 mM Tris-HCl, 1 mM EDTA, pH 8) and transferred to a microcentrifuge tube. 20 μl mutanolysin (1 mg/ml; Sigma, St. Louis, Mo.) was added, and the slurry was incubated at 37° C. for 1-2 hr while gently shaking/rotating. Following this, 50 μl 0.5 M EDTA, 50 μl 10% SDS and 50 μl 5 M NaCl were added with gentle mixing, followed by addition of 10 μl proteinase K (fresh 20 mg/ml or Sigma cat. no. P4850) and incubation at 37° C. for 1 hour. Subsequently, 50 μl sodium perchlorate (1 g/ml) was added to the mixture, followed by extraction with phenol:chloroform:isoamyl alcohol (25:24:1), and chloroform:isoamyl alcohol (24:1). Total DNA was then precipitated with 0.6 ml isopropanol and centrifugation. DNA pellets were washed briefly with 70% ethanol, dried and resuspended in 100-200 μl TE.

“Retransformation”

Plasmids that could not be observed directly in restriction enzyme digests of total genomic DNA (for example, due to low copy number or poor recovery) from Rhodococcus could be detected readily by electroporating an aliquot (typically 1-5 μl) of the total genomic DNA into competent E. coli cells, followed by selection on LB agar with appropriate antibiotics. This procedure requires that the plasmid in question is able to replicate in E. coli and it bears a suitable selectable marker. E. coli transformants recovered from these plates were cultured and plasmid DNA recovered via a miniprep procedure (e.g., Eppendorf Perfectpreps, Hamburg, Germany) and tested via restriction enzyme digestion and analysis. Finding a plasmid to be indistinguishable from the plasmid that had originally been introduced into the Rhodococcus cells confirmed the presence of the plasmid in the candidate Rhodococcus transformant.

Bacterial Conjugation

10 ml cultures of donor and recipient bacterial strains were cultured to late exponential stage in LB medium plus antibiotics, as appropriate. 0.5-1 ml from each donor strain was centrifuged briefly and the cell pellet was washed with 0.5 ml fresh LB (no antibiotics). The supernatant was discarded and 0.5-1 ml recipient cell culture was added to the washed donor cells and centrifuged briefly. The resulting mixed cell pellet was then resuspended in ca. 100 μl LB, and the cell slurry was dripped onto the center of a presterilized (with ethanol) mixed cellulose ester 0.2 μm filter (Millipore, Milford, Mass.), which lay on an LB agar plate (no antibiotic). The cell mixture was incubated overnight at 30° C., after which the cells were washed from the filter and resuspended with 0.5 ml fresh LB. Following resuspension, the cells were plated onto LB plates carrying the appropriate antibiotics and incubated for 2-8 days at 30-37° C. (depending on the plasmid being transferred and the tolerance of the recipient cells) until transconjugant colonies appeared.

Pulsed Field Gel Electrophoresis

Rhodococcus cells were embedded in low melting point agarose. Slices of embedded cells were incubated in bacterial lysis solution (10 mM Tris-HCl pH8, 50 mM NaCl, 100 mM EDTA, 0.2% sodium deoxycholate, 0.5% sarcosine, 1 mg/ml lysozyme) overnight at 37° C. Lysis buffer was replaced with digestion buffer (0.5M EDTA, 1% sarcosine, 0.5 mg/ml proteinase K) and incubated at 50° C. overnight. Slices of embedded cells were then loaded into the wells of a 1% agarose gel, 0.5×TBE (1×TBE=0.49M Tris-HCl, 0.49M boric acid, 0.001M EDTA; pH8), and run in a BioRad CHEF-DRII PFGE apparatus at 14° C. and 6 v/cm for 19 hrs, with a switch time beginning at 15 s and ramping to 75 s. Pulsed field gels were stained with ethidium bromide before photographing.

### Example 2

### Identification and Cloning of pB264

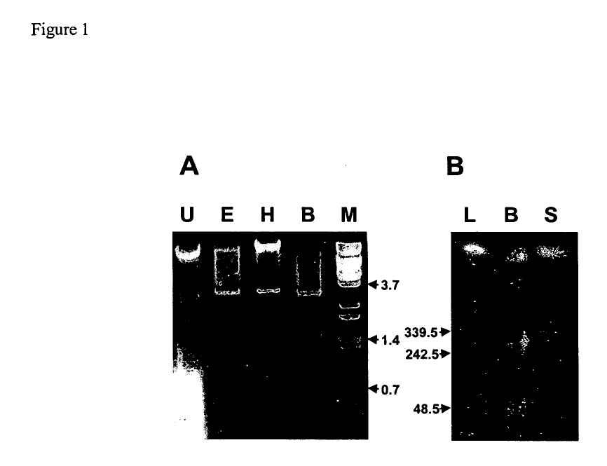

In an effort to develop plasmids for genetic manipulation of rhodococci, we examined a number of Rhodococcus strains for the presence of small, cryptic plasmids. Agarose gel electrophoresis of DNA isolated from the B264-1 strain revealed a ca. 5 kb plasmid, which we labeled pB264. Whereas neither EcoRI nor HindIII were able to cut this plasmid, digestion with BamHI produced three bands of ca. 3 kb, 1.5 kb and 0.4 kb (FIG. 1A). A sample of the undigested pB264 was isolated from an agarose gel via GeneClean. Isolation of circular DNA by this method produces a small amount of sheared material. We purified the sheared material from a second agarose gel, treated the DNA with Klenow fragment of DNA polymerase, and ligated the products into pCR-Script, producing pAL220. Sequencing of pAL220 revealed an insert of 4970 bp (GenBank accession AY297818). FramePlot analysis of the pB264 element revealed several ORFs. The two largest of these were ORF6 (729 nt) and ORF7 (933 nt). BLAST analysis of this sequence revealed that pB264 is nearly identical to pKA22, a plasmid isolated from R. rhodochrous NCIMB13064, which was sequenced but not further characterized. The sole exceptions to this alignment were a thymidine substitution at nt position 17 of pB264 (corresponding to the cytosine residue at nt position 3143 of pKA22) and a thymidine insertion at nt position 37 of pB264 (corresponding to a position between nt 3162 and 3163 of pKA22.) ORFs 6 and 7 of pB264 correspond to ORFs 1 and 2 of pKA22, respectively.

Discussion

The complete nucleotide sequence of the small, cryptic plasmid pB264 (4970 bp) of Rhodococcus sp. B264-1 was determined. The G+C content of the sequence of this plasmid is 64.8 mol %. pB264 proved to be identical to the largely uncharacterized cryptic plasmid pKA22 isolated from R. rhodochrous NCIMB13064 by Kulakov et al. The translational product of ORF7 (corresponding to ORF2 of Kulakov et al.) consists of 296 amino acids with an estimated molecular mass of approximately 33 KDa, whereas the 82 amino acid translation product of ORF8 has a predicted mass of approximately 9.2 KDa. BLAST analysis of ORF7 and ORF8 identified several additional database entries with greater than 50% of amino acid identity to the predicted gene product. These include the two components of a putative theta replicase (repA, encoding a putative primase, and repB, encoding a putative DNA binding protein) from Brevibacterium linens plasmid pLIN, as well as putative proteins encoded by the Mycobacterium fortuitum plasmid pAL5000 and relatives of this plasmid. These homologies suggest that the pB264 replicon belongs to the pAL5000 family of Gram-positive theta replicating plasmids, a group that includes plasmids that have been isolated from Bifidobacterium, Brevibacterium, Corynebacterium, Mycobacterium, and Neisseria. Furthermore, the deduced amino acid sequence of ORF7 contains the characteristic helix-turn-helix motif of DNA binding proteins. The presence of two major motifs within the RepA protein of pB264 that are characteristic of theta replicating plasmids provides additional evidence that pB264 uses a theta mode of replication. Our deletion analysis (as described below) of pB264 provides strong evidence that ORF7 and ORF8 are required for plasmid replication, supporting the designation of these ORFs as repA and repB. Further supporting this assertion is the observation that expression of repA and repB in trans supports replication of pB264 derivatives that lack either or both of these elements (FIG. 3). The RepA and RepB proteins of other plasmids from the pAL5000 and ColE2 families have also been shown to function in trans.

Currently, pB264 remains cryptic as no function, other than replication and mobilization functions, has been attributed to this plasmid. None of the predicted open reading frames, other than the designated repA and repB, show significant homology to any functionally defined sequence. In R. rhodochrous NCIMB13064, the pB264 homolog, pKA22, appeared to segregate with the loss of the ability to utilize short chain 1-chloroalkanes and the appearance of a naphthalene degrading phenotype, in that reversion to wild type phenotype appeared to correlate with disappearance of the cryptic plasmid. This association, however, is more likely attributed to the effects of plasmid integration into the chromosome rather than an explicit, plasmid encoded function.

### Example 3

### pB264 Replication

pB264 Replicates in Other Rhodococcus Strains

To test whether pB264 derivatives could be used as gene expression vectors, we sought to determine whether this plasmid could replicate in other Rhodococcus strains. Since ampicillin resistance is unsuitable for selection in Rhodococcus, we introduced additional markers for KanR, TsrR or GntR into pAL220, creating the plasmids pAL224, pAL231 or pAL298, respectively. Electroporation of these plasmids into strains B264-1, I24 or R. erythropolis SQ1 produced antibiotic resistant colonies. (Because the I24 strain has a high incidence of spontaneous resistance to kanamycin, pAL224 was not used to test replication in I24.) Unrearranged plasmids could be recovered from these transformants, indicating that each plasmid could replicate autonomously in these strains and that the pB264 element could support replication. The ColE1 replicon of pCR-Script itself does not appear to support replication in Rhodococcus as we have never been able to recover antibiotic resistant colonies when ColE1-derived plasmids were used to transform B264-1 or I24, regardless of the selectable marker (see also the deletion analysis of pB264, below.) The genetic diversity represented by these three strains among rhodococci suggests that pB264 derivatives should be able to replicate in a wide variety of Rhodococcus isolates.

Maintenance of pB264 is Temperature Sensitive

While Rhodococcus strain I24 can grow at temperatures above 40° C., the B264-1 strain does not grow significantly above about 32° C. To test whether replication of pB264 had a similar temperature limitation as its host bacterium, we introduced pAL298 (FIG. 2) into the I24 strain via electroporation. I24(pAL298) was grown in 10 ml LB+ gentamicin (10 mg/l) for 2 days at 30° C. 10 μl from this culture were diluted into six separate tubes, each containing 10 ml fresh LB without antibiotics. Three of these cultures were then incubated at 30° C., while the other three were incubated at 37° C. After two days, serial dilutions were prepared from each of these cultures and plated onto LB agar or LB agar supplemented with gentamicin. These plates were then incubated for three days at 30° C. to determine the proportion of bacteria that had retained the plasmid in each case. Results from these experiments are shown in Table. From the cultures that had been grown at 30° C., similar numbers of CFU were recovered on both the LB plates and the LB+ gentamicin plates, indicating that pAL298 was well maintained in the I24 strain at 30° C., despite the lack of antibiotic selection. In contrast, from the cultures that had been grown at 37° C., fewer than 0.06% of the CFU retained the antibiotic resistance markers, indicating that replication or maintenance of the plasmid was hindered at the higher temperature. This trend was observed with other derivatives of pB264 as well, regardless of the selectable marker employed.

TABLE 2
Colonies (CFU per ml) recovered from temperature sensitivity tests
of pB264 derivative.

Parent I24(pAL298) culture
grown in LB at:

| plated onto¹ | 30° C. | 37° C. |
|---|---|---|
| LB | 1.8 ± 0.2 × 10⁸ | 2.2 ± 0.7 × 10⁸ |
| LB + gentamicin | 1.1 ± 0.1 × 10⁸ | 1.3 ± 0.6 × 10⁵ |

¹composition of the medium used to test aliquots of serial dilutions; these plates were all incubated at 30° C.

In additional experiments, individuals were recovered from the few antibiotic resistant colonies that arose on the antibiotic plate (incubated at 30° C.) from a culture that had been grown at 37° C. Each of these colonies was streaked onto two fresh LB plates supplemented with antibiotics. One plate from each set was incubated at 30° C., while the other was incubated at 37° C. for two days. Of the 20 colonies tested in this manner, none grew well at 37° C., while all formed robust colonies at 30° C. This indicates that the few colonies that had retained the plasmids while the cells were growing at 37° C. were not the result of mutations that allowed replication at the higher temperature. More likely, these colonies represent the minority of the cells that retained a single copy of the poorly replicating plasmids during cell division at the higher temperature.

Identification of a Minimal Replicon of pB264

To identify the region within pB264 that is responsible for replication in Rhodococcus, we prepared a series of deletion derivatives of the plasmid pAL298. These deletions are depicted schematically in FIG. 3. The plasmid pAL298 contains the entire pB264 element, the ColEI origin, which does not by itself support replication in Rhodococcus sp. strains B264-1 or I24 (not shown), and a GntR marker.

Dozens of deletion derivatives were prepared from pAL298. These were first propagated in E. coli to recover a large amount of purified plasmid. Individual deletion derivatives were then introduced into Rhodococcus sp. I24 via electroporation. Rhodococcus sp. I24 was used as the host in these experiments, rather than Rhodococcus sp. B264-1 to decrease the possibility that some gene product in the host strain might be supporting plasmid replication in trans or via recombination with the native copy of pB264, which might confound the results. Following electroporation, cells were plated onto LB supplemented with gentamicin and incubated for 2-3 days at 30° C. to identify transformants. Colonies from these plates were inoculated into 10 ml of LB supplemented with gentamicin and grown for 2-3 days at 30° C. Total genomic DNA was recovered from each of these and examined for the presence of the intended plasmid either by direct restriction enzyme digest and agarose gel analysis or by the “retransformation” method (see Methods). These tests were necessary to confirm that GntR had not arisen spontaneously nor by recombination of the plasmids into the I24 genome.

The majority of deletion derivatives tested failed to produce colonies of transformed Rhodococcus sp. I24. Those capable of replicating in the I24 strain are depicted in FIG. 3. Examining the regions retained in plasmids 1-8 (FIG. 3) strongly suggests a role for the ORF7 and ORF8 gene products in replication of pB264. To test this hypothesis more directly, a 280 bp SphI fragment was deleted from the 5′ end of ORF7 (corresponding to the N-terminus of the encoded protein), creating plasmid 9 (FIG. 3). We were unable to obtain transformed colonies when electroporating plasmid 9 into Rhodococcus sp. I24, even though parallel electroporations of plasmid 8 were always successful. These results suggested that plasmid 9 had lost the ability to replicate autonomously in Rhodococcus. In contrast, when the gene product of ORF7 was supplied in trans, plasmid 9 could replicate in the I24 strain. This was accomplished by preparing plasmid 13 (pAL312), which can replicate in Rhodococcus sp. I24 because of the NG2 origin it possesses. ORF7 is positioned downstream of a constitutive promoter in this plasmid. pAL312 was introduced into the I24 strain, and competent cells were prepared from the transformant. Subsequently, plasmid 9 was introduced into I24(pAL312) by electroporation. Recovery of both plasmid 9 and pAL312 from the resulting transformants confirmed that each was replicating separately and that plasmid 9 was not being supported because of some recombination event. In contrast, plasmid 12, which expresses a truncated version of ORF7, was unable to support replication of plasmid 9, further confirming the role of the ORF7 gene product. In a similar series of experiments the region carrying ORF8 was deleted from either plasmid 8 or plasmid 6, creating plasmids 10 and 11, respectively. Neither of these plasmids produced colonies when electroporated into Rhodococcus sp. I24, unless that host was already carrying pAL314, which expresses both ORF7 and ORF8. Replication of plasmids 10 and 11 could not be complemented in trans by pAL312, indicating that ORF8 is required for replication. These results also indicate that all of the cis-acting elements required for plasmid maintenance (such as oriV) reside within the portions of pB264 that were retained in both plasmid 9 and plasmid 11, most likely within the 139 bp region that was retained immediately upstream of ORF7. While it seems likely that other elements may reside on pB264, such as copy number control elements, partitioning functions or plasmid addiction systems, we found that the smallest deletion derivative of pAL298 (plasmid 8, FIG. 3) was maintained as stably as pAL298 (if not more so) by Rhodococcus sp. I24 cells in both the presence and the absence of antibiotic selection (data not shown).

Discussion

The majority of small plasmids isolated from Gram-positive organisms are believed to replicate via a rolling circle mechanism, where the leading DNA strand is first synthesized from a starting nic site and then used as a template for production of the lagging strand. Unfortunately, many vectors based on RC origins of replication exhibit structural and/or segregational instability that is not observed in plasmids that employ a theta mode of replication. This instability appears to be exacerbated by the insertion of foreign DNA fragments. The observed instability of RC plasmids has been attributed to formation of single stranded DNA intermediates, which may result in deletions due to illegitimate recombination. Additionally, it has been proposed that the formation of high molecular mass multimers provides a selection against recombinant plasmids, thereby also encouraging the enrichment of deletion derivatives. The segregational instability of RC based plasmids containing cloned DNA sequences has similarly been attributed to this selection disadvantage. Rhodococcus has proven particularly refractile to gene cloning using such RC based vectors. By exploiting the theta-mode replication functions of pB264, shuttle vectors constructed will avoid the segregational and structural instability problems inherent to RC replicons. Our experience with pB264 derivatives supports this assertion.

The observation that replication or maintenance of pB264 is affected by the temperature at which the host cell is grown is reminiscent of the plasmid pSG5. Muth et al. discovered pSG5, a rolling circle type plasmid, in an isolate of Streptomyces ghanaensis. This isolate had a limited tolerance for elevated temperatures and could not grow above 35° C. While pSG5 derivatives could replicate in other Streptomyces isolates, it could do so only at lower temperatures (≦34° C.). Even though some of these strains could grow above 35° C. they could not retain the plasmid at this higher temperature. In the present experiments, the Rhodococcus sp. B264-1 strain cannot grow at 37° C. while the I24 strain can. Although pB264 could replicate in both strains, it was rapidly lost from the I24 strain when that host was grown at 37° C. (Table 2).

Three classes of theta replicating plasmids, designated A, B, and C, have been defined in the literature. These classes differ with respect to their structural features and are based almost exclusively on studies of Gram-negative bacteria. Theta replicating plasmids from Gram-positive bacteria have not yet been extensively studied, but most appear to fall into one of the first two classes. Gram-positive theta replicons, such as pAMβ1, have been identified that do not appear to fit into these established classes. Similarly, pB264 does not appear to fit easily into these theta classifications. The basic replicon of pB264 encodes RepA and RepB proteins essential for replication and does not appear to carry an oriA-like structure, which sets pB264 apart from the previously described Rep-dependent class A plasmids. Similarly, several features distinguish pB264 and class B replication. First, class B replicons do not encode Rep proteins, whereas pB264 does. Second, there is no homology between the pB264 replicon and the highly conserved ca. 600 nt elements that are characteristic of class B plasmids. Third, pB264 can replicate in at least three diverse species of Rhodococcus, while class B plasmids characteristically have a narrow host range. The pAL5000 family of replicons, and specifically pB264, superficially resemble class C replicons, consisting almost entirely of the E. coli-derived ColE2 and ColE3. Class C plasmids encode Rep proteins and do not require an oriA-like structure for replication. However, the homology between the replication regions of pB264 (or other pAL5000-type plasmids) and the class C plasmids is, at best, highly diverged. Furthermore, there is no significant homology between the Rep proteins of the pAL5000-type plasmids and ColE2. We, therefore, believe that pB264, and by extension the pAL5000 family of replicons, belongs to a separate class of theta replicating plasmids. Further work is required to determine how widespread this class is and what its relation is (if any) to the previously described class C replicons.

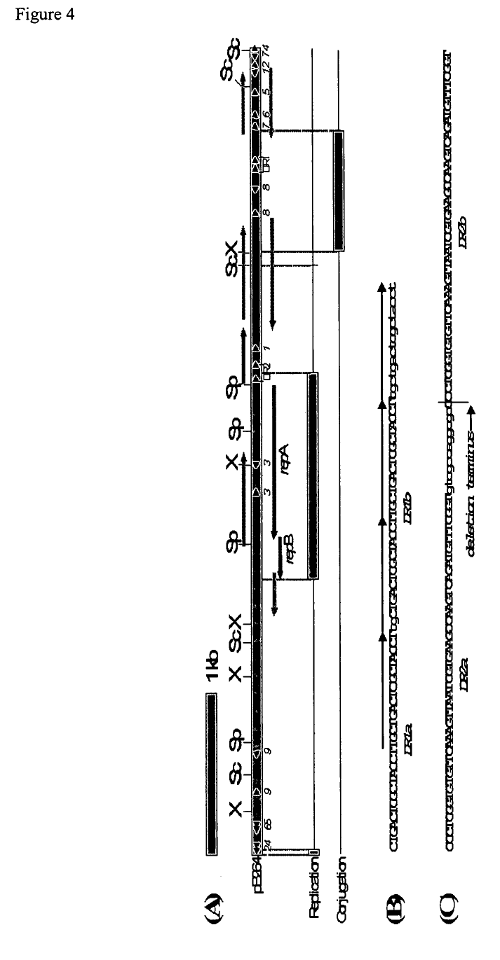

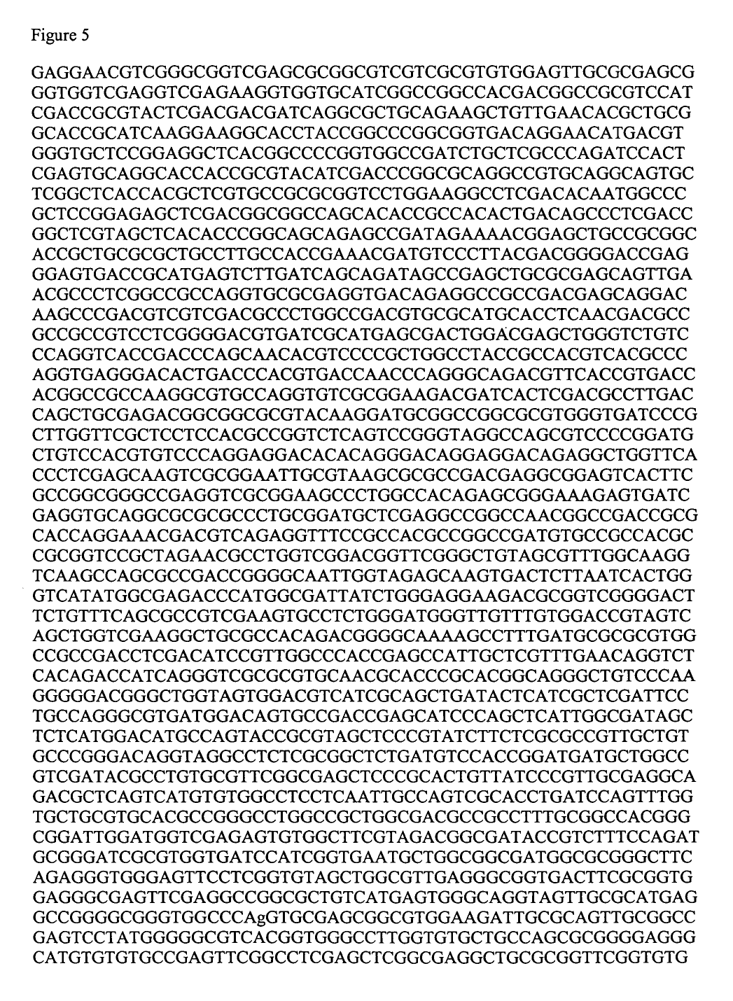

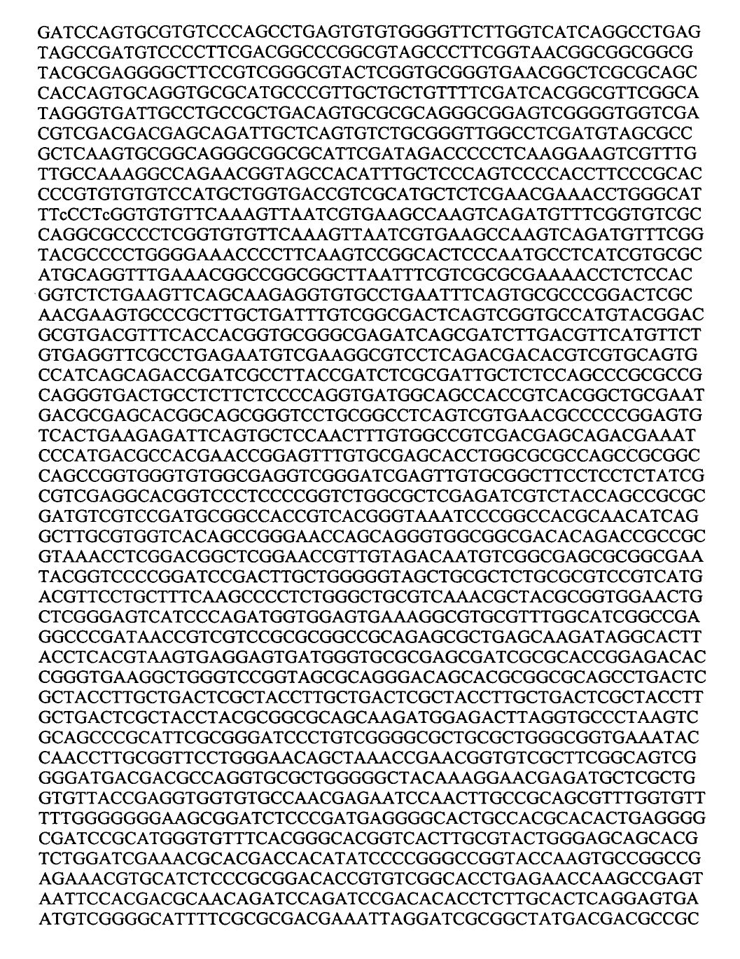

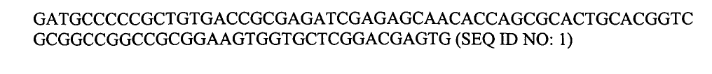

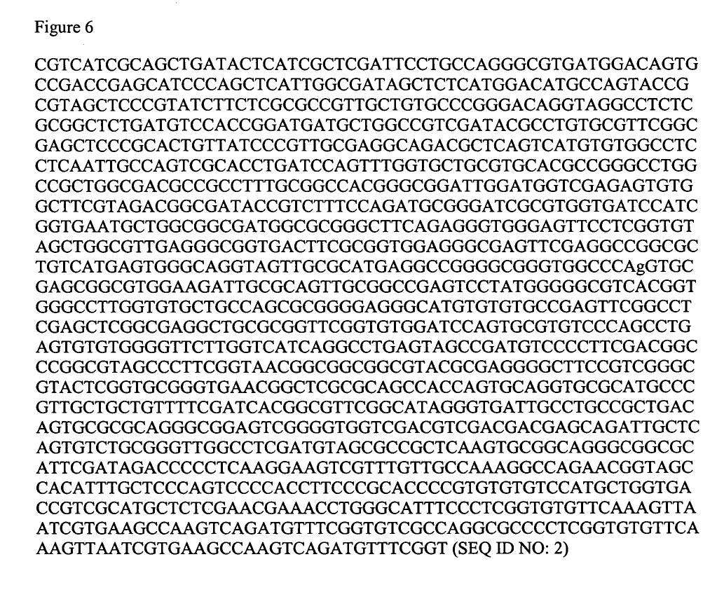

While replication functions in pAL5000 have been extensively studied, our results with pB264 suggest that this plasmid is part of a distinct sub-group. Experimental evidence has shown replication of pAL5000 to be dependent on several sequence domains immediately upstream of the repAB promoter. The “L-site” is a low affinity Rep binding site where replication initiates. The “H-site” contains GC-rich and AT-rich 8 bp palindromes, as well as two short, inverted repeats. Mutations within these structures interfered with binding of RepB as well as replication. pB264 contains no structures that resemble either the L-site or the H-site. However, pB264 does possess two AT-enriched domains, comprising DR2 (FIG. 4). Within this domain is a sequence that is highly conserved among pAL5000 and its relatives, although it has not been shown directly that the conserved element is required for replication of pAL5000. The smallest autonomously replicating derivative of pB264 (FIG. 3) retains only one of the two repeat elements within DR2. Chattoraj described a scenario in which two components of a direct repeat enabled copy number control, whereas only one copy was required for plasmid replication.

### Example 4

### pB264 in Bacterial Conjugation

pB264 Can be Mobilized Via Bacterial Conjugation

PFGE analysis of the Rhodococcus sp. B264-1 revealed the presence of at least two megaplasmids in the 250-300 kb size range (FIG. 1B). Megaplasmids have often been implicated in conjugal transfer of DNAs between bacterial cells. In many cases, these large extrachromosomal elements have been found to encode the functions (especially the trans acting components) necessary to physically conduct the DNA from the donor to the recipient cell. The presence of such elements in the B264-1 strain suggests the possibility that this strain can promote conjugal transfer of DNAs between cells. In other systems, small plasmids have been found or created that can also be transferred conjugally, though these small elements do not themselves encode the full complement of trans-acting functions required for transfer.

To test whether the plasmid pB264 could be mobilized from Rhodococcus sp. B264-1 into other Rhodococcus strains, we constructed the plasmid pXS9a, which carries the entire pB264 element, an E. coli origin of replication, and three selectable markers (KanR and TsrR for selection in Rhodococcus, and AmpR for selection in E. coli). This plasmid was introduced into the B264-1 strain via electroporation, and the presence of the unrearranged plasmid confirmed. (Examining plasmid DNA from these transformants revealed that establishment of pXS9a was accompanied by the loss of the native pB264, suggesting incompatibility of the two plasmids.) B264-1 (as a negative control) or B264-1(pXS9a) were then co-cultivated with prospective recipient strains as described in Methods. Following co-incubation, the cell mixtures were transferred to selective plates containing rifampicin (to select against the donor strain) and thiostrepton and kanamycin (to select for the plasmid) and incubated at 30° C. When R. erythropolis SQ1 or Rhodococcus sp. I24 R6 were used as recipients in these experiments, RifR, TsrR and KanR colonies were recovered that could easily be distinguished from the donor strain based on colony color and morphology. When plasmid DNA was isolated from these putative transconjugants, each was found to carry pXS9a, unrearranged.

Transconjugants were also recovered when B264-1 R1 was used as a recipient strain. In contrast, no RifR, TsrR and KanR colonies were recovered in these experiments when recipient cultures were co-incubated with untransformed donors (B264-1), heat-treated donors [B264-1(pXS9a) incubated at 65° C. for 20 minutes], filtered supernatants from B264-1(pXS9a) cultures, or naked pXS9a plasmid DNA. These results indicate that the pB264 derivative pXS9a can be transferred conjugally from Rhodococcus sp. B264-1 to other Rhodococcus strains. We have used conjugation to successfully transfer other pB264 derivatives from Rhodococcus sp. B264-1 to a variety of other Rhodococcus strains, including an environmental isolate of R. erythropolis that is distinct from the SQ1 strain, and three other environmental isolates of R. ruber, R, rhodochrous and R. fasciens.

Given that the Rhodococcus sp. I24 strain can grow at higher temperatures than can the B264-1 donor strain, we sought to exploit temperature as a selective agent in such conjugation experiments. To accomplish this, we first needed a version of the pB264 plasmid that could replicate stably at the elevated temperature. Rather than identifying a mutant pB264 with this property, we chose to simply add a second, temperature stable origin of replication to the pB264 element, creating the plasmid pJANET (FIG. 2; the NG2 origin from Corynebacterium diphtheriae replicates in both E. coli and Rhodococcus, and is stable at 37° C.). We found that growth at 37° C. (in conjunction with gentamicin) is very effective for selecting transconjugants when the donor is B264-1 (pJANET) and the recipients are the I24 strain or other Rhodococcus strains that grow at 37° C.

To estimate the efficiency of transfer, conjugations were carried out between B264-1(pJANET) and the I24 strain. Following co-incubation of the donor and recipient strain (prior to the selection step), serial dilutions were prepared from the cell mixture. Portions from these dilutions were plated onto either LB agar or LB agar supplemented with gentamicin, and these plates were treated in three different ways (Table 3). (1) Incubating the LB gentamicin plates at 37° C., conditions that only permit growth of I24(pJANET), we were able to identify the total number of transconjugants per experiment. (2) Incubating the LB gentamicin plates at 30° C., we were able to calculate the total number of donor cells [B264-1(pJANET)] used in each experiment. Under these conditions, both donors and transconjugants would be able to grow. However, the transconjugants have an easily distinguishable phenotype (color) and the total number of donors greatly exceed the total number of transconjugants, so it was a simple matter to determine which colonies were derived from donor or recipient cells. (3) From LB plates (no antibiotic) grown at 37° C., we were able to determine the total number of recipient cells used in the experiment. Under these conditions, no donors can grow, but all recipients can grow, regardless of whether they have acquired the antibiotic resistance marker. From six replicates of this experiment, representing 2 sets of donor and recipient cultures, three independent matings each, and then serial dilutions prepared from each of these matings, we calculated that 1 transconjugant was generated per 1.11±0.28×10⁵ donors. This corresponds to 1 transconjugant per 1.37±0.36×10⁶ recipients.

TABLE 3
Frequency of conjugal transfer of pB264 derivatives from Rhodococcus
sp. B264-1 to Rhodococcus sp. I24.

| Plating Condition | Description | # CFU per ml |
|---|---|---|
| LB + gentamicin, 30° C. | Donors | 3.51 ± 0.78 × 10⁸ |
| LB, 37° C. | Recipients | 4.32 ± 0.98 × 10⁹ |
| LB + gentamicin, 37° C. | Transconjugants^{a} | 3.17 ± 0.37 × 10³ |

^{a}Several of the transconjugants were subsequently tested, and the presence of unrecombined pJANET was confirmed in each of these I24 derivatives.

Origin of Transfer

Trans-acting elements are likely to play an important role in the conjugal transfer of the plasmid from B264-1 to other Rhodococcus strains. Given the size of pB264 and the presence of megaplasmids in the B264-1 strain, it is entirely possible that none of the required trans-acting factors are encoded by pB264. However, it is almost certain that a specific cis-acting element (an origin of transfer or oriT) resides on pB264. To determine what portion of pB264 enables conjugal transfer in cis, we prepared a series of deletions in pJANET and tested each for the ability to be transferred in conjugations between the B264-1 donor and an I24 recipient (using growth at 37° C. and gentamicin for selection of transconjugants). We were unable to recover transconjugants with the majority of deletion derivatives tested in this manner, including pAL281, which is related to pJANET, but lacks the entire pB264 element. B264-1 donors carrying the plasmids pJANET XΔ27 (plasmid 17 of FIG. 3), pJANET XS (plasmid 18), pJANET XB (plasmid 19), or pAL315 (plasmid 20) produced transconjugants at frequencies similar to B264-1(pJANET). In all cases, transconjugants were tested further and found to have taken up the unaltered test plasmids. Sequences in common between the smallest of these plasmids, pJANET XS and pAL315, correspond to a region of 743 bp from pB264 (FIG. 3) that is apparently devoid of ORFs.

Discussion

Bacterial conjugation is a specialized gene transfer process that involves unidirectional transfer of DNA from donor to recipient bacteria by a mechanism that requires cell-to-cell contact. Plasmids spread both within and between bacterial species primarily through conjugal transfer. Plasmids are classified according to their conjugative ability as conjugative (auto-transmissible), mobilizable (transmissible only in the presence of a helper conjugative plasmid), or non-mobilizable. Conjugative plasmids contain the necessary genetic information to catalyze conjugative DNA processing and DNA transport. Mobilizable plasmids lack part of this machinery. Naturally occurring mobilizable plasmids contain oriT, the only DNA sequence required in cis for conjugation, and they may encode a set of proteins to process oriT and make it available to the transport complex.

Plasmid pB264 and genetically labeled derivatives could be transferred to different Rhodococcus strains by conjugation, showing that pB264 is a mobilizable plasmid. Experiments with filtered culture supernatants, heat-treated donor cultures and naked DNA distinguished conjugal transfer from other possible modes of DNA transfer such as phage-mediated or direct DNA uptake. A region involved in mobilization of pB264 was identified that is distinct from the regions involved in replication. This region includes both inverted repeats (IR8) and direct repeats (DR1) that may serve as the oriT sequence (FIG. 4). Others have proposed that such repeat elements play a role in conjugal plasmid transfer.

### Example 5

### Generating a nimA Knockout Mutant in Rhodococcus

Introduction

The role of nimA in the indene bioconversion in Rhodococcus sp. I24 was investigated. NimA is one of the genes, located on the ˜50 kb element, that is believed to encode the small subunit of the enzyme responsible for the NIM activity. This gene was knocked out in Rhodococcus sp. KY1 and the resulting mutant was phenotypically characterized.

Materials and Methods

Key bacterial strains and plasmids are presented in Table 1. Additional strains and plasmids are described in the text. Bacteria were routinely cultured in LB medium except where noted; Medium Rare was used as the defined medium (per liter: 1.4 g (NH₄)₂SO₄; 1 g MgSO₄.7H₂O; 0.015 g CaCl₂.2H₂O; 1 ml A9 trace elements solution (per liter: 0.5 g FeSO₄.7H₂O; 0.4 g ZnSO₄.7H₂O; 0.02 g MnSO₄.H₂O; 0.015 g H₃BO₃; 0.01 g NiCl₂.6H₂O; 0.25 g EDTA; 0.05 g CoCl₂.6H₂O; 0.005 g CuCl₂.2H₂O; filter sterilize; stored at 4° C.); 1 ml stock solution A (per liter: 2.0 g NaMoO₄.2H₂; 5.0 g FeNa-EDTA; filter sterilize and stored at 4° C.); 35.2 ml 1 M phosphate buffer (per liter: 113 g K₂HPO₄; 47 g KH₂PO₄); and 40 g glucose. For preparation of Medium Rare (NH₄)₂SO₄, MgSO₄.7H₂O and CaCl₂.2H₂O were added to 863 ml of water and autoclaved. Then sterile Stock solution A, A9, 1M phosphate buffer and 100 ml of 400 g/l glucose solution were added.) Antibiotics were included in media (as noted) at the following concentrations: gentamycin, 10 μg/ml; streptomycin, 50 μg/ml; rifampicin 10 μg/ml; kanamycin, 100 μg/ml. Fine chemicals were obtained from Sigma (St. Louis, Mo.) except where noted.

Additional media used in the invention include: MB Liquid Medium (per liter: 5 g yeast extract; 15 g bacto tryptone; 5 g bacto soytone; and 5 g NaCl).

Methods are as described in Example 1. Additional methods are presented below.

The primers used in this invention are listed and described in Table 4.

TABLE 4
Oligonucleotide PCR primers

| Name | Sequence (5′-3′) | Anneals to (in 5′-3′ orientation) |
|---|---|---|
| KO1 | CAC ACC TAA ACT GAC ATG CT | Rhodococcus sp. I24 genomic DNA |
| KO2 | GCA ATA TGG TCT TCG CCT | Rhodococcus sp. I24 genomic DNA |
| KO3 | CGA GAT CGA GAG CAA CAC CA | pAL302 nt 5066-5085 |
| KO4 | CGT TGT AAA ACG ACG GCC A | pAL302 nt 5552-5534 |
| hpNIMAUP | AAA AAA CAT ATG AGC ACA | Rhodococcus sp. I24 genomic DNA |
| | TCG ACG TCG ACG ACG ACC | |
| hpNIMADO | AAA AGG ATC CTC AGC CGG | Rhodococcus sp. I24 genomic DNA |
| | CGA GGG TGA GCC GGC CG | |

Analysis of Metabolite Intermediates by HPLC

The consumption of indene and the accumulation of oxygenation products were observed by reversed-phase high performance liquid chromatography (HPLC) analysis. Cultures were grown by inoculating one colony of either KY1 or the mutant strains into 250 ml flasks containing 50 ml LB. 250 ml cultures were inoculated to an OD600 of 1.0. After reaching an OD₆₀₀ of 3.0, the cultures were divided to three 250 ml flask. To each flask, containing 50 ml of culture, either indene, toluene or naphthalene was added to 2 mM final concentration for preinduction. After the flasks were incubated at 37° C. for one hour, pure indene was added to a final concentration of 2 mM. Cultures were incubated at 37° C. with an agitation of 225 rpm for 90 hours.

Samples for HPLC analysis were prepared by removing 500 μl aliquots from each culture and adding 500 μl of an 1:1 mixture of isopropanol and acetonitrile to the sample. Samples were mixed, centrifuged for 10 minutes at 13,000 rpm and filtered through a 0.22 μm filter. 20 μl were applied to a Zorbax RX-C8 column (5 μm particle, 4.6 mm i.d.×25 cm, Agilent Technologies, Inc.; Andover, Mass., USA) using a gradient method. The HPLC system was of the 1050 series (Agilent Technologies, Inc.; Andover, Mass., USA) The UV absorbtion of the eluent was monitored at 220 nm, and the intermediates were identified and quantified by comparison of the retention time to pure standards. The solvent system consisted of solvent A (filtered MilliQ water) and solvent B (acetonitrile).

Growth on Naphthalene as a Sole Carbon Source

Mutant and wildtype Rhodococcus sp. KY1 and I24 strains were grown in medium rare liquid culture containing naphthalene (2 mM) as a sole carbon source. Strains were incubated at 30° C. and sampled for about 5 days. The OD_{600 nm} of each sample was measured using a Spectronic®20 Genesys™ spectrophotometer (Thermo Spectronics, Rochester, N.Y.).

Southern Analysis

Southern analysis was performed using the DIG High Prime DNA Labeling and Detection Starter Kit II for Chemoluminescent Detection with CSPD® (Roche Molecular Diagnostics, Indianapolis, Ind.) as described by the manufacturer. The nimA probe was generated from Rhodococcus I24 genome DNA via PCR (using primer NIMAUP and NIMADO) and labeled as described above. Genomic DNA of the mutants, as well as wildtype genomic DNA and the plasmid pAL302 as controls, were digested with restrictions endonucleases SnaB I and Cla I and separated using conventional electrophoresis (2.2.10) before blotting. DNA was transferred to a positively charged nylon membrane (Roche Molecular Diagnostics). Hybridizations were performed in Standard Hybridization Buffer as recommended by Roche Molecular Diagnostics [5×SSC, 0.1% (w/v) sodium-lauroyl-sarcosine, 0.02% (w/v) SDS, 1% Blocking Reagent (provided with the kit); 1×SSC is 0.15M NaCl, 0.015M sodium citrate] at 68° C. Hybridized membranes were washed twice for 15 minutes at 42° C. with gentile agitation. Chemiluminescent detection was performed before exposure to X-ray film.

Results

To investigate the roles of the nimA and nimB genes in indene bioconversion in Rhodococcus sp. I24 a gene knock out strategy was applied. Rhodococcus sp. KY1, a derivative of I24, was the strain selected for the knock out strategy. KY1 lacks an approximately 340 bp element and a toluene inducible dioxygenase activity, however, KY1 contains a smaller extra chromosomal 50 kb element, which is believed to carry the naphthalene inducible mono- and dioxygenase genes. Flux analysis experiments have shown that the naphthalene inducible dioxygenase activity is down regulated in KY1 cells. Thus, knocking-out the gene responsible for naphthalene inducible monooxygenase activity in KY1 should result in the loss of indene conversion activity in this strain. The gene nimA is believed to encode the small subunit of the naphthalene inducible monoxygenase, and is similar to the small subunit of a styrene monooxygenase from Pseudomonas sp. VLB120.

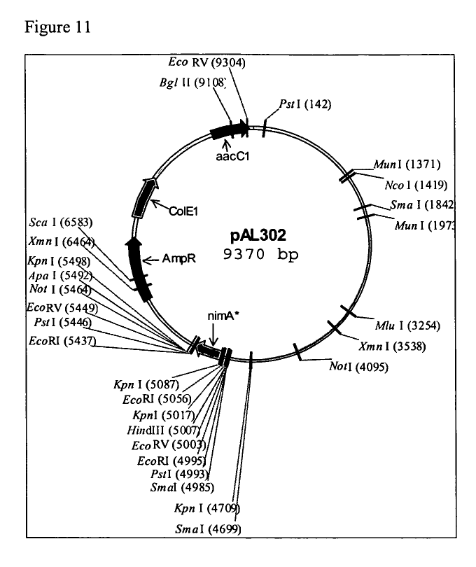

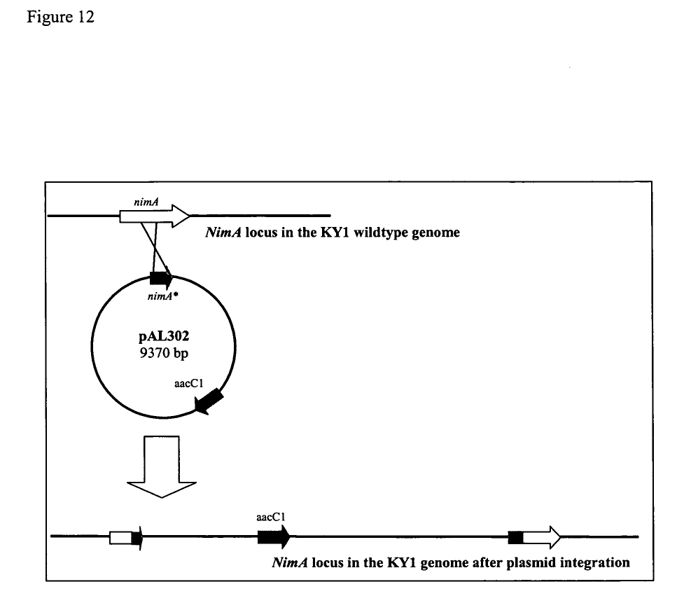

Mating experiments were carried out to generate a knockout of nimA in KY1 via homologous recombination. Rhodococcus sp. KY1 was used as the recipient strain and Rhodococcus sp. B264-1 containing plasmid pAL302 (FIG. 11) was used as the donor strain. Plasmid pAL302 carries an internal part of the nimA gene, allowing the insertion of this plasmid into the genome of KY1, by homologous recombination at the side of the nimA gene. Since this plasmid only contains an internal part of nimA, the insertion causes a knock out of the intact nimA gene (FIG. 12). The gentamicin resistance marker of the plasmid was used for selection.

Further, pAL302 is temperature sensitive; it can replicate in Rhodococcus sp. B264-1 at 30° C., but not in the recipient Rhodococcus sp. KY1, if it is grown at 37° C. As the B264-1 strain does not grow on temperatures higher than 30° C., and pAL302 is not replicated at 37° C. in KY1, only recombinant strains of KY1 that integrated the plasmid into the genome, would be expected to grow on LB plates in the presence of gentamicin at 37° C. About twenty independent yellowish to orange pigmented gentamicin resistant transconjugants were grown. Four or them, designated 5, 7, 10 and 17, were isolated and characterized more in detail.

Characterization of the Genotype of the Knock Out Candidates by PCR

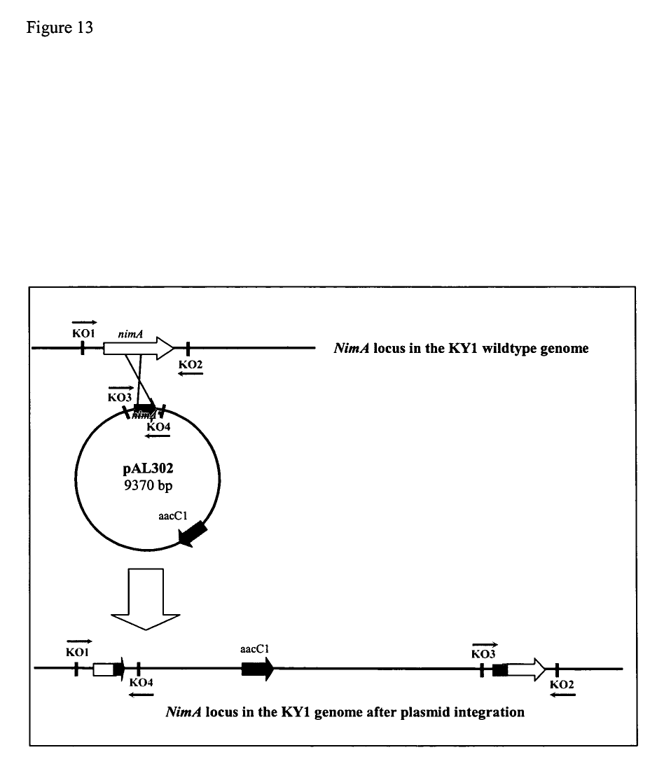

To verify the integration of the plasmid pAL302 into the nimA gene by homologous recombination in the mutants, four different primers were designed (see Table 4). Used in different combinations, these primers can be used to monitor the integration of pAL302 into the nimA locus. Primers KO1 and KO2 were designed to amplify a PCR product (˜670 kb) containing the intact nimA gene from the KY1 genome (FIG. 13). KO3 and KO4 anneal to pAL302 DNA flaking the internal fragment of nimA (nimA *) and should amplify a ˜527 kb sized product (FIG. 13). In the knock out mutant strain both of these combinations should not result in any PCR product under the chosen PCR conditions. Whereas the primer pairs KO1/KO4 as well as KO2/KO3 should result in PCR products of different sizes after annealing to the recombinant genomic DNA (FIG. 13), but should not yield a PCR product with the wildtype genomic DNA.

Genomic DNA of four potential knock out mutants (mutants 5, 7, 10 and 17) was isolated and monitored for homologous recombination by PCR. PCR analysis of mutant 5 resulted in products only when primer combinations KO1/KO2 and KO3/KO4 were used, indicating that the nimA * fragment did not integrate into the genome. This mutant strain was not further analyzed.

Mutants 7, 10, and 17 yielded PCR products with all primer combinations, indicating that pAL302 integrated into the nimA locus. However the functional nimA of the wildtype genome and the unintegrated plasmid pAL302 were also present in both mutants, signaling that these mutants possess a ‘mixed phenotype’.

Elimination of the Functional nimA Gene Copies in the Mutants

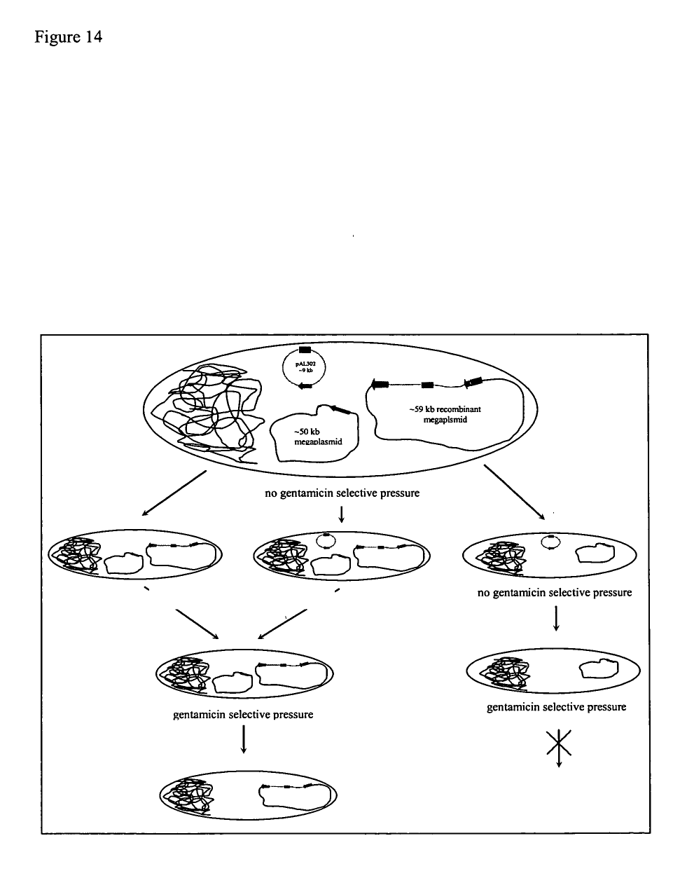

As mutants 7, 10, and 17 still contained copies of the intact wildtype nimA gene and unintegrated pAL302, it was concluded that these mutants possessed a ‘mixed genotype’. Thus, the following strategy was employed to yield a homogenous genotype, exhibiting only the mutant nimA gene (FIG. 14).

We concluded that the selection with the gentamicin marker did not work, because plasmid pAL302 was replicated in these mutants even at 37° C. This was unexpected and our first step was to eliminate the unintegrated plasmid pAL302.

After loosing the autonomous plasmid the gentamicin resistance would be only sustained by the pAL302 DNA integrated in nimA. Therefore, the mutants were plated on LB plates without antibiotics at 37° C. Under those conditions the plasmid pAL302 would not be under selective pressure and the mutant strains should loose the plasmid, since initially the replication of pAl302 was temperature sensitive and did not occur above 30° C.

After three passages the mutants were plated onto LB plates, now containing gentamicin again. This step should select mutants with the recombinant megaplasmid containing the plasmid pAL302 with the gentamicin resistance marker integrated in nimA, whereas in the wildtype megaplasmid no gentamicin resistance marker exists.

After four passages of cultivation on LB agar plates containing gentamicin, colonies from the initial mutants 7, 10, 17, 7-1, 7-2 and 7-3 were purified. The plasmid segregation experiments were repeated in liquid cultures. Cells were first cultivated in liquid LB medium without antibiotics as well as in liquid LB medium containing gentamicin. Then aliquots of those cultures were cultivated in liquid LB medium with (indicated by ++ or −+) as well as without gentamicin (indicated by +− or −−). The genomic DNA of cells from the last cultures was isolated and again analyzed via PCR as described above.

Verifying the Genotype of the Mutants

From the plasmid segregation experiments 18 different strains were obtained, which were further analyzed. Genomic DNA from each strain was isolated and used as template DNA in PCR reactions applying the primer pairs used above.

The PCR analysis of the different mutants showed that in 7 of 18 strains, the primer combination KO1/KO2 did not yield a PCR product, indicating the loss of the intact nimA gene. Controls show the expected bands, the nimA * from pAL302 DNA and nimA from KY1 DNA.

To confirm the loss of the intact nimA gene southern blot analysis was performed on the genomic DNA samples of mutants selected by PCR. To use the nimA gene as a probe, the gene was amplified via PCR applying genomic DNA of KY1 as template DNA and primers hpNIMAUP and hpNIMADO (Table 4). The DIG labeled nimA probe hybridized to the nimA gene in the wildtype genome as well as in the recombinant genome and to the nimA * portion of plasmid pAL302.

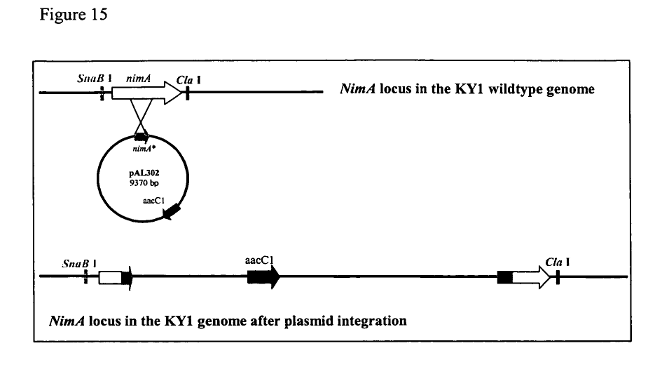

The restriction site for SnaB I is located approximately 1.9 kb downstream of the nimA gene and the restriction site for Cla I is located approximately 700 bp upstream (FIG. 15). If the whole plasmid pAL302 was integrated in the genome of the KY1 strain, the expected band size in the southern blot was approximately 12.5 kb. From the wildtype genome the expected band size was approximately 3.2 kb.

In the genomic DNA of all of the six tested mutant strains a band larger than 10 kb was detected. The expected band size was about 12.5 kb. However, the resolution of the agarose gel in this size range is too low for exact size determination, and the size of this band was estimated. Most significantly Southern blot analysis of the mutant strains showed confirmed that the loss of the intact nimA gene (the approximately 3.2 kb band) in the mutant strains 7++, 7+−, 7-1++, 7-2++, 7-2+−, 7-3++.

Phenotypical Characterization of the Mutants 7++ and 7+− With Respect to Their Indene Conversion Ability

To establish whether there is a difference between the mutants and the wildtype phenotype with respect to the ability to metabolize indene, a shaking flask experiment was performed. The mutant strains 7++ and 7+− and the wildtype strain KY1 were grown in shaking flasks containing LB medium and their indene conversion ability was analyzed as described above. The cultures were induced by addition of either indene, toluene or naphthalene to a final concentration 2 mM, and after one hour indene (2 mM final concentration) was added. The indene conversion by these cultures was monitored over 91 hours or until the indene concentration reached a minimum. The occurrence and concentration of intermediates was determined by HPLC analysis of culture supernatants as described above.

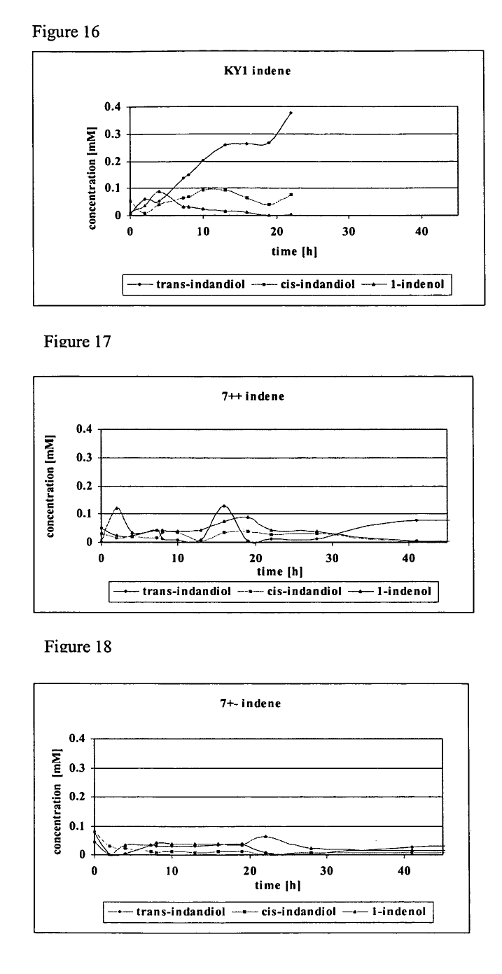

In FIGS. 16 to 24 the indene conversion profiles of the wildtype strain KY1 and the two mutant strains 7+− and 7++ under three different inductions conditions are shown. The exact determination of the indene concentration during the experiment was not possible because indene is as a hydrophobic compound not soluble in water. Therefore the course of the indene concentration during the experiment is not included in FIGS. 16 to 24. However, the tendency and time course of the indene concentration decrease was qualitatively determined.

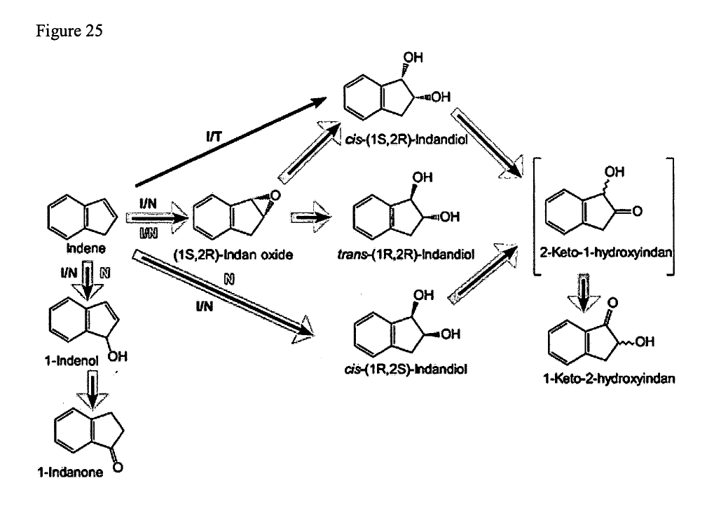

The KY1 strain was able to convert indene. In the cultures which were induced only with indene, the indene concentration decreased and reached the minimum after 12 hours. The concentration of 1-indenol increased in the first 4 hours, then it went down and reached a minimum after 24 hours. The cis- and trans-indandiol concentration increased during the whole time course of the experiment. The trans-indandiol concentration reached about 0.38 mM during the 22 hours lasting experiment and the cis-indandiol concentration reached a maximum of 0.09 mM after 10 hours. The cis-indandiol concentration subsequently decreased, most probably because it was further converted to keto-hydroxyindan (FIG. 25).

The indene induced cultures of the mutant strains 7++ and 7+− behaved different from the KY1 control culture. In the cultures of both mutants the indene concentration went down very slowly and reached the minimum after 40 hours. They also show no significant increase of the metabolite concentrations over a time range of 91 hours, which indicated at least the partially loss of the indene conversion ability.

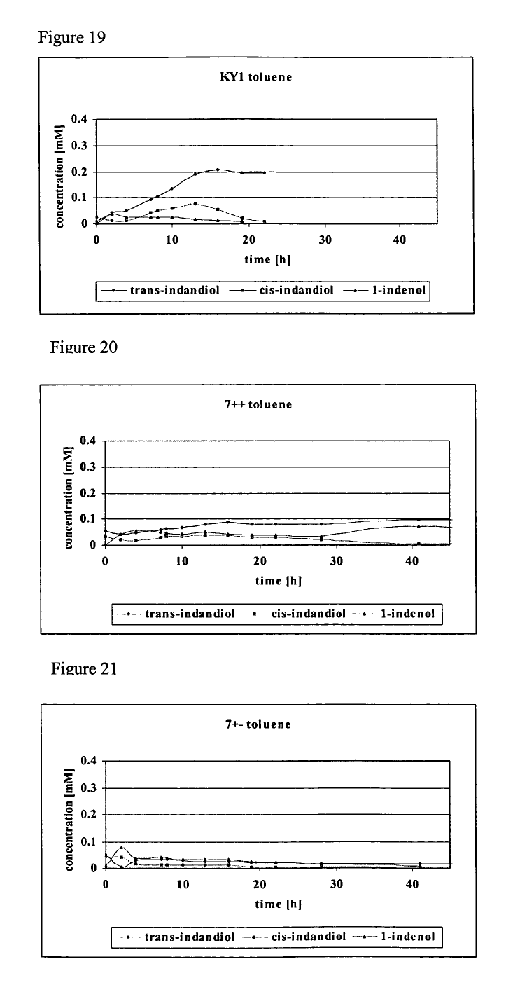

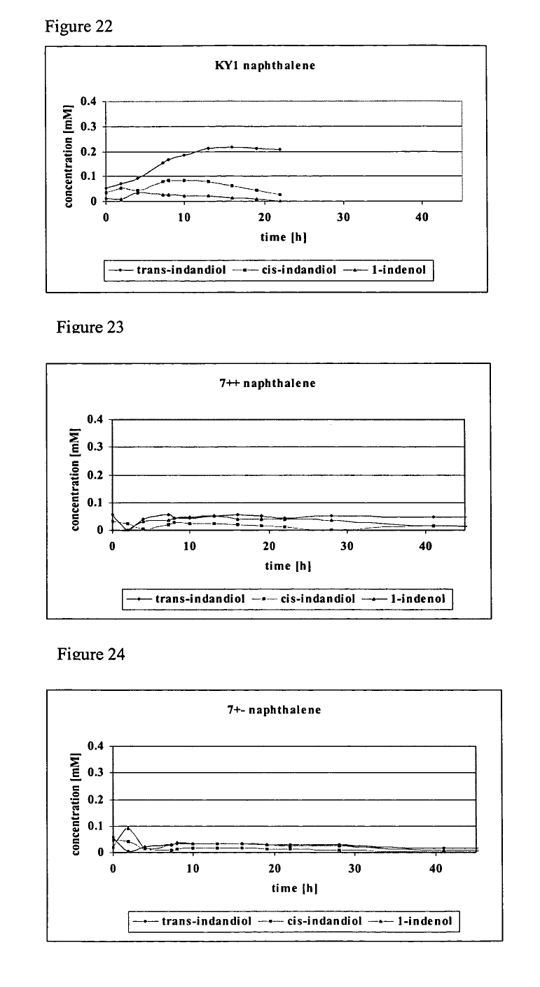

Similar results were obtained in the cultures induced with toluene (FIGS. 19-21) and naphthalene (FIGS. 22-24). The indene concentration in the KY1 culture decreased faster than in the mutant cultures. The cis- and trans-indandiol concentrations increased in the wildtype culture, whereas in the mutant strains no significant increase was recorded.

To verify the stability of the genotype of the mutants and the control strain during this experiment, genomic DNA was isolated after the shaking flask experiment from cells of each culture. The DNA was used as template in PCR analysis.

Verifying the Stability of the Genotypes of Mutants 7++ and 7+− During the Indene Conversion Experiments

To verify the stability of the knock out of nimA in the mutant strains 7++ and 7+− PCR analysis with the different primer combinations was performed. Genomic DNA was isolated from cells of every culture taken at the start of the shaking flask experiment and after the experiment had ended. These DNA samples were used as template DNA. PCR analysis of the mutant strains 7++ and 7+− on both time points resulted in products when primer pairs KO3/KO4, KO1/KO4 and KO2/KO3 were used which indicates the presence of the autonomous pAL302 as well as the recombinant megaplasmid. No products were obtained using primer combination KO1/KO2, indicating the absence of the wildtype megaplasmid, carrying the intact nimA gene.

PCR analysis of the KY1 genomic DNA samples resulted in only one product. Only primer pair KO1/KO2 worked, which indicated the presence of the wildtype megaplasmid.

Phenotypical Characterization of the Mutants 7++ and 7+− With Respect to Their Ability to Grow on Naphthalene as a Sole Carbon Source

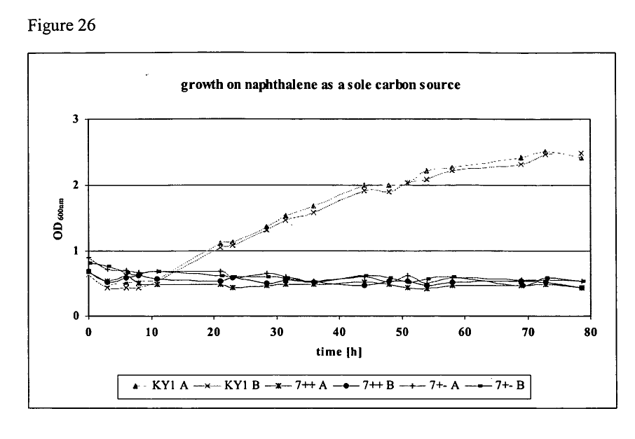

As the wiltype strain Rhodococcus sp. KY1 was able to use naphthalene as a sole carbon source, it was of interest to establish the behavior of the mutant strains 7++ and 7+− grown in medium rare containing naphthalene. The different strains were grown as described above in shaking flasks in medium rare containing naphthalene as sole carbon source. In the analysis of the OD_{600 nm} of the different cultures over 5 days an increase of the OD_{600 nm} in the wildtype strain Rhodococcus sp. KY1 was detected (FIG. 26). In the mutant strains 7++ and 7+− no change of the OD_{600 nm} was detected, which indicates that those strains were not able to grow on naphthalene as a sole carbon source.

Discussion

Rhodococcus I24 was previously shown by Chartain et al. to oxygenate indene via at least three independent pathways: a naphthalene inducible dioxygenase (NID), a toluene inducible dioxygenase (TID), and a naphthalene inducible monooxygense (NIM). Treadway et al. cloned a series of genes from the I24 strain, which they called nid operon because it apparently encodes enzymes for the NID pathway. Genetic and phenotypic as well as sequence and homology evidence suggested the identification of operons that contribute to both the TID and NIM system.

Genetically, the ability to degrade toluene and to convert indene to cis-1S-2R-indandiol (which reflect activity of the TID system) is only found in the strains that contains the tid genes. The TID system was expressed in E. coli and indene conversion activity in the crude protein extract was detected.

Southern analysis have shown that the KY1 strain, a derivative of the I24, has lost both the tid gene and the ability to grow on toluene. Xian O'Brien found, that the tid genes reside on a 340 kb element in the I24 strain, and that this element was lost from Rhodococcus KY1. In contrast, the same studies revealed that the nim and nid operons reside on a smaller, ˜50 kb extrachromosomal element. Both the I24 and KY1 strains have this element, and both have the NIM and NID activities.

At the DNA sequence level, both the tid and the nim operons show sequence homology and gene organizations that are consistent with their suspected functions. The first four ORFs of the tid operon have been expressed in E. coli. In in vitro assays using the protein extract, indene conversion activity was detected.

In this invention, the NIM system was of interest and the involvement of nimA in the indene bioconversion was shown. A gene knock out strategy was applied in Rhodococcus sp. KY1, which lacks the ˜340 kb element as well as the toluene inducible dioxygenase activity. However KY1 contains the ˜50 kb megaplasmid, which is believed to carry the naphthalene inducible mono- and dioxygenase genes. In the KY1 strain indene can be oxygenated via these two systems (NIM and NID).

Yanagimachi et al. found that the primary pathway of indene conversion in KY1 is through the monooxygenase enzyme that is responsible for converting over 94% of the indene taken up to (1S,2R)-indan oxide. It was also shown, that the dioxygenase pathway plays only a minor role in the indene bioconversion of Rhodococcus. Therefore it was expected to loose the indene bioconversion activity in KY1 by generating a knock out of the monooxygenase genes which are involved in the indene bioconversion process.

NimA shows a high degree of similarity to the small subunit of a styrene monooxygenase from Pseudomonas sp. VLB120, and is believed to encode the small subunit of the enzyme responsible for the NIM activity. Therefore it was the target for the gene knock out experiment in this invention.

A knock out strategy, based on a homologous recombination was applied. The nimA gene was interrupted by the plasmid pAL302 in the mutant strains 7++ and 7+−, which was verified by PCR and Southern blot analysis. The phenotypical characterization of those two mutant strains with respect to their indene conversion ability, was performed in a shaking flask experiment. The mutants 7++ and 7+− showed as expected a completely different metabolic profile in respect to the indene conversion. Both mutant strains lost the ability to convert indene into cis- and/or trans-indandiol. In the wildtype strain Rhodococcus sp. KY1 the same results as reported by Yanagimachi et al. were detected; indene was converted into both cis- and trans-indandiol.

The knock out of the nimA gene resulted in a loss of the indene conversion ability of the generated mutants, which leads to the conclusion, that the nimA gene encodes at least a subunit of the monooxygenase enzyme. At the same time we found that the mutant strains 7++ and 7+− cannot metabolize naphthalene sufficiently to support growth, however the wildtyp strain Rhodococcus sp. KY1 is able to grow on naphthalene as a sole carbon source.

Future work focused on restoring the nimA activity in the mutants, and second and third the knockouts of the other known open reading frames designated as nimB and ORF5468, to determine their role in the indene bioconversion and the NIM system. Also analysis of the gene products of nimA, nimB and ORF5468, expressed in E. coli and likely purified using Ni-NTA columns are of interest. Especially in respect to their indene oxygenation ability, but also the investigation of interactions between the different gene products using SELDI analysis could give more useful information about the genes corresponding to the NIM system.

### Example 6

### nimB and ORF5468 in Rhodococcus Strain KY1 Function in Naphthalene Metabolism

Introduction

Rhodococcus strain KY1 is able to grow on naphthalene as a sole carbon source via two pathways-a naphthalene-inducible dioxygenase system and a naphthalene-inducible monooxygenase system. We investigated the roles of two genes suspected to operate in naphthalene metabolism, nimB and ORF5468, by knocking out the genes. Derivatives of Rhodococcus B2641 carrying knockout plasmids were mated with wildtype KY1, and the transferred plasmids were able to integrate into the targeted KY1 genes by single crossover homologous recombination, disrupting nimB and ORF5468. The nimB and the ORF5468 gene knockout strains lost the ability to utilize naphthalene as a sole carbon source, suggesting that both genes play critical roles in naphthalene metabolism. Sequence homology data suggests that nimB is a ring-cleavage catechol dioxygenase and that ORF5468 is a hydroxylase; both types of enzymes have been implicated in naphthalene catabolism. The sequence homology data in conjunction with the naphthalene growth experiment results lead us to believe that nimB and ORF5468 encode naphthalene catabolic enzymes. The elucidation of nimB and ORF5468's function will be useful for genetically manipulating Rhodococcus KY1 for use in bioremediation and enantiomerically pure chiral syntheses.

Materials and Methods

All important bacterial stains and plasmids used in this study are described in Table 1. Rhodococcus and E. coli were grown in Luria Bertani (LB) broth containing 5 gg/ml gentamicin (Sigma, St. Louis, Mo.) when selecting for plasmid-carrying strains. LB plates containing 2% agar were prepared and contained 10 μg/l gentamicin as appropriate. Rhodococcus KY1 and E. coli were grown at 37° C. unless otherwise noted. Rhodococcus B264-1 was grown at 30° C.

Minimal media plates for Rhodococcus KY 1 contained 2% agar, 10 pg/ml gentamicin, 1.4 g/l (NH4 2SO4, 1.0 g/l MgSO4.7H2O, 0.015 g/l CaCl2.2H2O, 2.0 g/l NaMoO4.2H2O, 5.0 g/l FeNa.EDTA, 0.5 g/l FeSO4.7H2O, 0.4 g/l ZnSO4.7H2O, 0.02 g/l MnSO4-H₂O, 0.015 g/l H3BO3, 0.01 g/l NiCl2-6H2O, 0.25 g/l EDTA, 0.05 g/l CoCl2.6H2O, 0.005 g/l CuCl2.2H2O, 113 μl K2HPO4, and 47 g/l KH2PO4. Medium was sterilized by autoclaving. As a carbon source, 500 μl of 40% sucrose were spread onto minimal media plates, or 10-15 naphthalene crystals were added to the lid of up-side-down minimal media plates.

Table 5 provides a list of primers used for sequencing and PCR.

TABLE 5
Primers used for sequencing and PCR

| Number | Primer Name | Sequence (5′ to 3′)^{a} | Comments |
|---|---|---|---|
| 1 | pAL298 5′ | CGAGATCGAGAGCAACACCA | Anneals 5′ to gene in pARF50 |
| | | | and pAR51 |
| 2 | pAL298 3′ | GCAAGGCGATTAAGTTGGGT | Anneals 3′ to gene in pAR50 |
| | | | and pAR51 |
| 3 | nimB forward | CGAAGACCATATTGCTCACCGA | Anneals 5′ to nimB in KY1 |
| | | | genome |
| 4 | nimB reverse | GGACTCGAAGATCATCTCGT | Anneals 3′ to nimB in KY1 |
| | | | genome |
| 5 | ORF5468 forward | CGTACTGGTTGAACTGGGT | Anneals to 5′ end of ORF5468 |
| | | | in KY1 genome |
| 6 | ORF5468 reverse | GGAGAAGATCGTCGATGTCGT | Anneals to 3′ end of ORF5468 |
| | | | in KY1 genome |

^{a}Primers were ordered from Integrated DNA Technologies (Coralville, IA)

Methods are as described in Example 1. Additional methods are provided below.

PCR was performed using the Qiagen (Valencia, Calif.) HotStarTaq kit according to the manufacturer's instructions. Primers are listed in Table 2. A 50 μl PCR reaction consisted of approximately 20 ng template DNA, 1 IiM each of forward and reverse primers, 200 μM dNTP mix (Invitrogen, Carlsbad, Calif.), 1× Q-solution (Qiagen), 1× PCR buffer (Qiagen), 2.5 units HotStarTaq (Qiagen), and sterile filtered water to volume. Reactions were carried out in a DNA Engine Pltier Thermal Cycler (PTC-200) frrm MJ Research (Rneo, Nev.) as follows: 95° C. for 15 minutes, 95° C. for 1 minute, 58.5° C. for 2 minutes, 72° C. for 3 minutes (steps 2-4 were repeated 25 times), 72° C. for 10 minutes, followed by 4° C. hold

For plasmid rescue from Rhodococcus KY1, total genomic DNA was isolated from Rhodococcus as described above, and approximately 1 pg of DNA was digested with the Sal I restriction enzyme. The restriction enzyme was then heat inactivated at 65° C. for 20 minutes. To the 10 μl restriction enzyme reaction, 10 μl of ligation mix was added (2 μl T4 ligation buffer, 0.5 μl T4 ligase, 7.5 μl H2O), and the ligation was incubated at 16° C. overnight. 5 μl of ligation reaction was transformed into chemically competent XLI-Blue E. coli.

For true breeding experiments, Rhodococcus KY1 gene knockout candidates were grown in liquid LB gentamicin for at least one week. The cells were pelleted, washed, and grown in LB without antibiotic selection for at least 48 hours. Equal dilutions of the KY1 gene knockout culture grown in LB were plated on an LB gentamicin plate and an LB plate. Colonies were counted on both plates, and the numbers were compared.

For Naphthalene growth experiments, Rhodococcus gene knockout strains and control starins were streked on minimal media plates containing gentamicin. Naphthalene-containing plates were incubated in an environmental jar. Seven days after streaking the plates, bacterial growth on naphthalene as a sole carbon source was compared to growth on sucrose as a sole carbon source.

BLAST analysis (www.ncbi.nlm.nih.gov/) reveals that the nimB amino acid sequence shares 94% amino acid identity with the Rhodococcus sp. I edoD/I t-gene, which encodes a type II (catechol) dioxygenase. ORF5468 shares approximately 34% amino acid identity with many styrene monooxygenases and phenol hydroxylases, including the large subunit of the two-component phenol-2-hydroxylase I component A in Bacillus thermoglucosidasius A7.

Results

To knockout the nimB and ORF5468 genes in Rhodococcus KY1, we mated plasmid-carrying derivatives of B264-1 with KY1 (Table 6). The orange KY1 potential gene knockouts appeared on the experimental selection plates as spherical colonies, with each of the three replicate matings generating between 1 and 10 candidate knockouts.

TABLE 6
Conjugal transfer pairs for generating targeted nimB and
ORF5468 gene knockouts.

| Donor | Recipient | Description |
|---|---|---|
| B264-1 (pAR50) | KY1 | Knock out nimB gene in KY1 |
| B264-1 (pAR51) | KY1 | Knock out ORF5468 gene in KY1 |
| B264-1 | KY1 | Negative control-no plasmid transferred |
| B264-1 (pAL298) | KY1 | Negative control-no homology for |
| | | recombination |
| B264-1 (pJANET) | KY1 | Positive control-plasmid replicates |
| | | without integrating into genome |

Several of the candidate nimB and ORF5468 knockout colonies were selected at random inoculated into LB gentamicin, and grown at 37° C. Twenty-one potential knockouts were inoculated, but only 13 grew in liquid culture. The potential nimB knockouts were named 50A1, 50A2, 50A4, 50B2, 50B3, 50C3, and the potential ORF5468 knockouts were named 51A1, 51A2, 51A3, 51A4, 51B1, 51C2. Two additional potential ORF5468 knockouts, KY1 (pAR51) a and KY1 (pAR51)b, were also generated by following the same mating protocol

Using genomic DNA isolated from the potential knockouts, we isolated rescued plasmids containing the drug resistance markers. Total genomic DNA was cut with Sal I, which has a restriction site 3′ to the E. coli origin of replication, GentR marker, and AmpR marker in the plasmid, and sites near the 5′ end of the nimB and ORF5468 genes in the KY1 genome. The resulting DNA fragments were self-ligated with T4 DNA ligase, and the ligation mixture was transformed into supercompetent XL1-Blue cells. E. coli transformants containing the drug resistance marker were selected in liquid LB gentamicin. Plasmids were isolated by minipreparation and sequenced using the pAL298 5 primer (Table 5). Sequencing results of plasmids rescued from the nimB candidate knockouts were aligned with the full-length nimB gene and the pAR50 plasmid sequence using BLAST 2 Sequences on the NCBI website (www.ncbi.nlm.nih.gov). Likewise, the sequencing result from of plasmids rescued the candidate knockouts were aligned with the full-length ORF5468 gene and with the pAR51 plasmid. The plasmid rescue sequencing results are shown in Table 7. Rescued plasmids that shared more than 373 by identity with nimB or more than 438 by identity with ORF5468 most likely came from targeted gene knockout strains.

TABLE 7
Sequencing data for plasmids rescued from potential nimB
and ORF5468 knockouts

| Potential | |
|---|---|
| Knockout | |
| Strain | Plasmid Rescue Status/Results |
| nimB | |
| target | |
| 50A1 | 1025 bp identity with pAR50, 373 bp identity with nimB |
| 50A2 | 541 bp identity with pAR50, 850 bp identity with nimB |
| 50A4 | Not done |
| 50B2 | 1025 bp identity with pAR50, 373 bp identity with nimB |
| 50B3 | 541 bp identity with pAR50, 828 bp identity with nimB |
| 50C3 | 1025 bp identity with pAR50, 373 bp identity with nimB |
| ORF5468 | |
| target | |
| 51A1 | 1006 bp identity with pAR51, 438 bp identity with ORF5468 |
| 51A2 | 1006 bp identity with pAR51, 438 bp identity with ORF5468 |
| 51A3 | Not done |
| 51A4 | 1006 bp identity with pAR51, 438 bp identity with ORF5468 |
| 51B1 | 1006 bp identity with pAR51, 438 bp identity with ORF5468 |
| 51C2 | 526 bp identity with pAR51, 873 bp identity with ORF5468 |
| KY1 | 1006 bp identity with pAR51, 438 bp identity with ORF5468 |
| (pAR51)a | |
| KY1 | 1006 bp identity with pAR51, 438 bp identity with ORF5468 |
| (pAR51)b | |

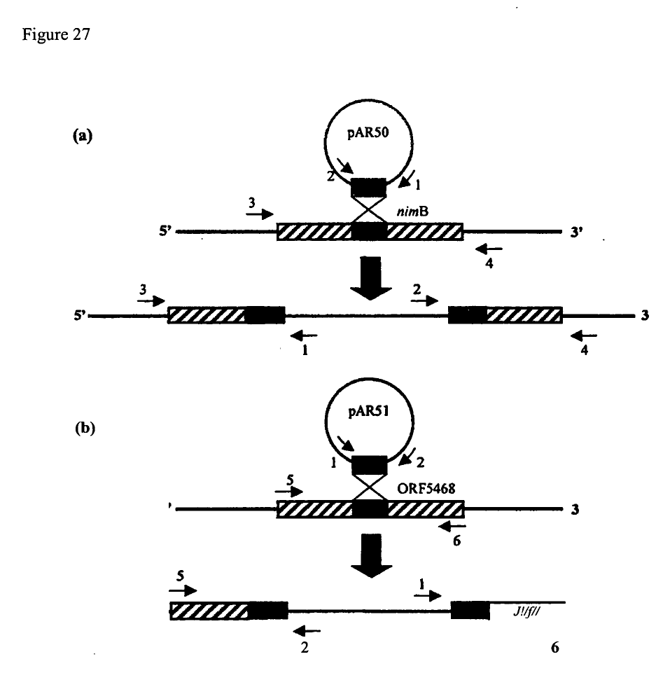

After obtaining positive results from the plasmid rescue sequencing, we confirmed the knockouts using PCR. FIG. 27 shows the recombination events for generating the nimB and ORF5468 knockouts as well as the primer combinations used to verify the knockouts. The expected band sizes from the diagnostic PCR are shown in Tables 8 and 9 for the nimB and ORF5468 knockout candidates respectively.

TABLE 8
Expected sizes of PCR products (base pairs) in KY1 nimB knockout
candidates with and without integration of the plasmid by homologous
recombination.

Primer Combinations

| | 1&2 | 3&4 | 1&3 | 2&4 |
|---|---|---|---|---|
| No recombination | 704 | 1000 | NPE^{a} | NPE |
| Recombination | NPE | NPE | 969 | 1060 |

^{a}No Product Expected

TABLE 9
Expected sizes of PCR products (base pairs) in KY1 ORF5468
knockout candidates with and without integration of the plasmid
by homologous recombination.

Primer Combinations

| | 1&2 | 5&6 | 1&6 | 2&5 |
|---|---|---|---|---|
| No recombination | 771 | 1312 | NPE | NPE |
| Recombination | NPE | NPE | 969 | 1060 |

Promising nimB knockout candidate 50A2 was subjected to a true breeding experiment. 50A2 maintained its gentamicin resistance phenotype after being grown at 37° C. in the absence of gentamicin for 48 hours. The aliquot of LB liquid culture that was plated on LB agar produced 201 colonies, and the equal-volume aliquot plated on LB gentamicin agar produced 173 colonies. This is a 14% difference in colony yield.

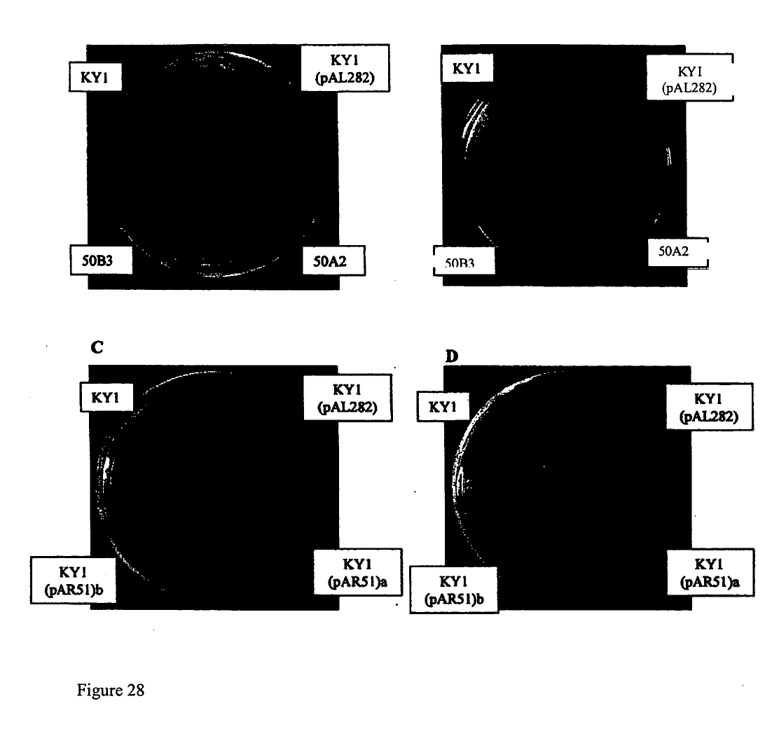

After verifying the knockouts by plasmid rescue and PCR, we tested their ability to grow on naphthalene as a sole carbon source. Knockout candidates were streaked on minimal media plates with the KY I (pAL282) positive control (wildtype genome and gentamicin resistance plasmid) and the KY 1 negative control (no gentamicin resistance). The results are shown in FIG. 28. Note that there is a low-level of growth for strains 50A2 and 50B3 on naphthalene, whereas KY 1 (pAR51)a and KY 1 (pAR51)b do not appear to grow at all on naphthalene. When KY1 pAL282 was grown on naphthalene as a sole carbon source, we observed a brownish-orange by-product in the agar and on the walls of the desiccator jar. This compound is presumably a quinone byproduct from naphthalene metabolism.

Discussion

Using a targeted gene knockout technique, we have elucidated the functions of nimB and ORF5468 in Rhodococcus KY 1. In addition to sequence homology data, the results from a previous genetic analysis of dioxygenase enzymes in Rhodococcus I24 (Treadway et al., 1999) led us to hypothesize that nimB and ORF5468 are involved in naphthalene metabolism. We chose to study the naphthalene degradation pathway in Rhodococcus KY1 as opposed to the parent strain I24, because KY1 lacks the toluene-inducible dioxygenase system present in I24. The cross-reactivity between the naphthalene-inducible dioxygenase system and toluene-inducible dioxygenase system for aromatic hydrocarbon substrates makes I24 undesirable for studying only naphthalene catabolism. KY1, which lost a 340-kb megaplasmid encoding the toluene-inducible dioxygenase, is a much cleaner organism for examining the enzymes of the naphthalene-inducible monooxygenase and dioxygenase systems.

Self-replicating and conjugationally transferable Rhodococcus plasmids pAR50 and pAR51_(“knockout plasmids”), containing a temperature sensitive origin of replication, were employed in Rhodococcus mating to knockout the target genes nimB and ORF5468 by single-crossover homologous recombination. Knockout candidates were selected for gentamicin resistance encoded by the plasmid at the nonpermissive plasmid replication temperature. Previous researchers have identified conjugation genes on Rhodococcus plasmids, and our pAL298 plasmid also carries genes allowing for transfer between strains. Insertion mutagenesis by homologous recombination of a temperature sensitive plasmid containing a drug resistant marker has been demonstrated as an effective technique to knockout genes and select for mutants in E. coli. Combining the tools of conjugal plasmid transfer, homologous recombination by a single crossover event, and a temperature sensitive plasmid with antibiotic resistance marker, we have attempted to create nimB and ORF5468 gene knockouts. Our knockout technique is different in its use of conjugal transfer instead of electroporation to transfer the knockout plasmid. Making electrocompetent KY 1 cells involves a laborious, time consuming, and inefficient technique, and we avoid this process by utilizing the cells' natural ability to share plasmids.

To verify knockouts of targeted genes, we had the most success with the plasmid rescue technique. Sequencing data from the nimB knockout candidates 50A2 and 50B3 show at least 828 by of continuous alignment with the nimB gene, and ORF5468 knockout candidates KY I (pAR5I)a and KY1 (pAR51)b show at least 873 by of continuous alignment with the ORF5468 gene (Table 7). The continuous alignment extended to the end of the sequencing reaction results, and although we did not attempt to sequence any further the rest of the nimB gene presumably resides beyond the region of accurate sequencing that can be reached with the primers that we used. These data indicate that the 373 by of nimB on pAR50 and the 438 bp fragments of ORF5468 on pAR51 recombined with the full length genomic copies of the respective genes. In contrast to the positive plasmid rescue results from the aforementioned knockouts all of the other knockout candidates gave us negative results. Alignment of the other nimB and ORF5468 rescued plamids with the appropriate gene showed only 373 or 438 by homology (Table 7), which is the size of the respective gene gragments that were inserted in the pAL298 knockout vector. In addition, these rescued plasmids aligned perfectly with their knockout plasmids, suggesting that we are only recovering the un-recombined knockout plasmids. We believe that there was no homologous recombination in any knockout candidates other than 50A2, 50B3, KY1 (pARS1)a and KY1 (pAR51)b.

After obtaining the positive plasmid rescue results with 50A2, 50B3, KY 1 (pARS1)a and KY1 (pAR51)b, we confirmed their nimB and ORF5468 knockout status using PCR. Because there are homologous regions from pAR50 and pAR51 plasmids that integrated into the genome, the knockout strains could be releasing their integrated plasmids at a low frequency via homologous recombination. Unexpected bands suggest that there is wildtype nimB in the nimB knockouts and wildtype ORF5468 in the ORF5468 knockouts. Again, these results suggest that the knockouts are losing their plamsids at a low frequency. It may be that these plasmid excision even are happening at a low rate detectable only by PCR but the resulting strains are not visable due to 10% of the growth marker. The double banding patter in lane B, the negative control, was seen in all PCR products with KY1 and primers 1 & 2. With the positive plasmid rescue and encouraging PCR results, we are confident in calling Rhodococcus KY1 50A2 and 50B3 nimB knockout strains and KY1 (pAR51)a KY1 (pAR51)b ORF5468 knockout strains.

Furthermore, 50A2 breeds true in the absence of gentamicin selection, suggesting that the integrated plasmid is stably maintained. The 14% difference in colonies grown on the LB plate versus the LB gentamicin plate is statistically insignificant For instance, if one copy of the target gene were knocked out of a two-copy plasmid, we would expect 50% fewer colonies on the LB gentamicin plate in comparison to the LB plate. Because the nimB and RF5468 knockout plasmids contained gene inserts with approximately the same number of base pairs homologous to the KY1 genome, we would expect the nimB and ORF 5468 knockout strains to recombine out their knockout plasmids at rougly the same frequency. We therefore conclude that both the nimB and the ORF5468 knockouts are stable.

We hypothesized that nimB and ORF5468 are involved in naphthalene metabolism because of previous experiments that examined another gene in the ORF5468-nimRABC gene cluster and because of our BLAST analysis for homologous sequences in GenBank. A nimA gene knockout strain failed to grow on naphthalene, and the mutant phenotype was rescued when nimA was added on a plasmid. Perhaps other genes in this cluster also play a role in naphthalene metabolism. Because the nimA and nimB genes lie so close together in the Rhodococcus KY 1 genome, they are probably co-expressed on the same transcript, which suggests that nimB is also involved in naphthalene metabolism. Furthermore, BLAST analysis of the nimB gene showed significant homology to catechol dioxygenases, which act downstream in the naphthalene degradation pathway, and ORFS468 showed significant homology to hydroxylases and monooxygenases, which may catalyze several steps in the naphthalene degradation pathway.

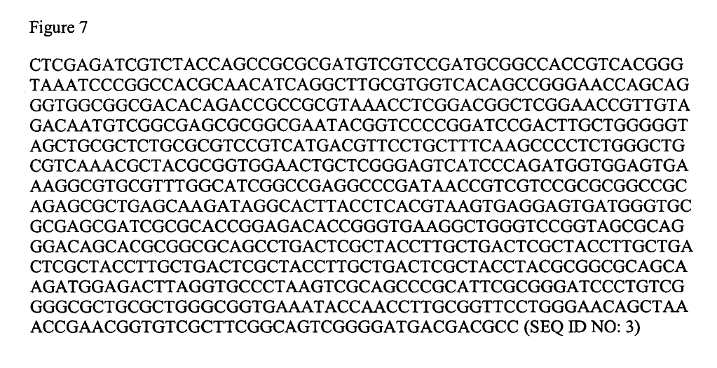

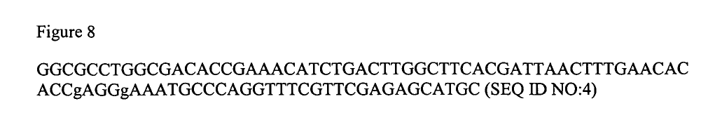

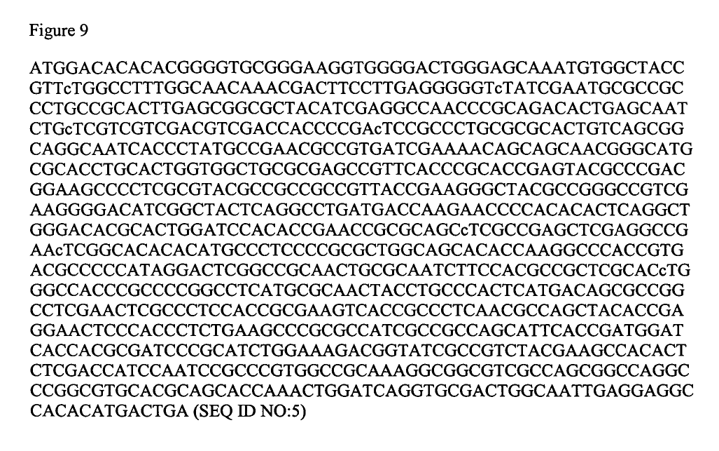

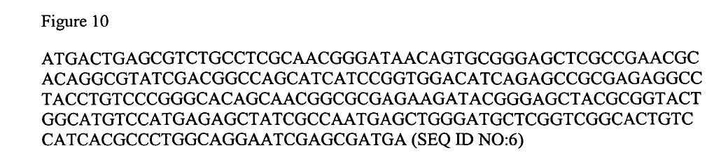

To characterize the nimB and ORF5468 gene knockouts' ability to utilize naphthalene as a sole carbon source, we conducted a naphthalene growth experiment. The results in FIG. 7 show that both the nimB and ORF5468 knockouts' growth is severely retarded when naphthalene is the only carbon source, further supporting our hypothesis that nimB and ORF5468 function in the naphthalene degradation pathway. We are currently conducting a complementation study to see whether the nimAB genes added in traps can rescue 50A2 and 50B3's inability to proliferate on naphthalene, and we are also transforming KY I (pAR51)a and KY I (pAR51)b with an ORF5468 expression plasmid.

We propose several explanations for the low level of 50A2 and 50B3 growth on the minimal media plus naphthalene plates, as opposed to no growth. First and foremost, it is important to realize that KYI possesses two naphthalene degradation pathways-one begins with a naphthalene-inducible dioxygenase and the other begins with a naphthalene-inducible monooxygenase. It is quite possible that we knocked out a gene in one of the pathways, while the other pathway was still functional. However, the naphthalene-inducible monooxygenase and the naphthalene-inducible dioxygenase pathways converge after the first two or three enzymatic steps, and we may be knocking out an enzyme shared by both pathway It is possible that another catechol dioxygenase in KY1 may be prtially redundant with nimB, giving our knockouts the ability to grow very slowly on naphthalene. A thrid possibility is that nimB resides on a plasmid present in multiple copies.

We could be knocking out one copy of the genes while there is a functional copy left in the cells; however, the true breeding experiments suggest that this is not the case. Finally, it is possible that metabolic products from our KY1 (pAL282) positive control could be diffusing in the agar and supplying a carbon source to the gene knockout strains. As can be seen in FIG. 7, KY1 (pAL282) produces brown quinones when grown on naphthalene, and the nimB knockouts could be using these organic compounds as a carbon source. We are carrying out another naphthalene growth experiment to evaluate this possibility by streaking each knockout on its own plate.

Although we have two nimB and ORF5468 gene knockouts in-hand, we experienced low rates of homologous recombination in our mating experiments. Integration of plasmids into bacterial chromosomes by single crossover homologous recombination has been extensively studied in Lactobacillus sake. The researchers showed that plasmid integration frequency is logarithmically proportional to the extent of homology between 0.3 and 1.2 kb, where 0.3 kb homology was the lower limit allowing for significant single crossover recombination Because our knockout plasmids only contain 373 and 438 by of homology, we probably would have seen higher knockout rates if larger pieces of the nimB and ORF5468 genes had been cloned into pAL298.

In Rhodococcus KYI, nimB and ORF5468 catalyze steps in the naphthalene degradation pathway. nimB most likely encodes a catechol dioxygenase in the naphthalene degradation pathway, and ORF5468 probably encodes a hydroxylase or monooxygenase in the pathway. Further work is needed to elucidate the exact function of ORF5468 and nimB in KYi. Because the indene bioconversion pathway in Rhodococcus KY 1 has been extensively characterized (FIG. 25), we plan to grow the nimB and ORF5468 knockouts with indene and analyze the products by high pressure liquid chromatography. Hopefully, this additional data will enable us to determine exactly which steps the nimB and ORF5468 gene products catalyze in the naphthalene metabolic pathway.

Equivalents

The present invention provides among other things novel compositions and methods for genetic manipulation of Rhodococcus bacteria. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

All publications and patents mentioned herein, including those items listed below, are hereby incorporated by reference in their entirety as if each individual publication or patent was specifically and individually indicated to be incorporated by reference. In case of conflict, the present application, including any definitions herein, will control. Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: Bell K S et al., J Appl Microbiol 1998, 85(2):195-210; O'Brien X M et al., Appl Microbiol Biotechnol 2002, 59(4-5):389-399; Vogt Singer et al., J Bacteriol 1988, 170(2):638-645; Shao et al., Lett Appl Microbiol 1995, 21(4):261-266; Hashimoto et al., J Gen Microbiol 1992, 138(Pt 5):1003-1010; Desomer et al., Applied and Environmental Microbiology 1990, 56:2818-2825; Dabbs et al., Plasmid 1990, 23(3):242-247; Quan et al., Plasmid 1993, 29(1):74-79; Dabbs et al., Mol Gen Genet 1988, 211(1):148-154; De Mot et al., Microbiology 1997, 143(Pt 10):3137-3147; Zheng et al., Plasmid 1997, 38(3):180-187; Voeykova et al., Biotechnology Letters 1994, 16(6):555-560; Chartrain et al., J Ferment Bioeng 1998, 86(6):550-558; Ishikawa et al., FEMS Microbiology Letters 1999, 174:251-253; Altschul et al., Nucleic Acids Res 1997, 25(17):3389-3402; Kulakov et al., Plasmid 1997, 38(1):61-69; Thomas et al., Mol Microbiol 2000, 37(3):485-491; Snyder et al., Molecular genetics of bacteria. Washington, D.C.: ASM Press; 1997; Simon et al., Bio/Technology 1983, 1:784-791; Bevan et al., Nucleic Acids Res 1984, 12(22):8711-8721; Zhang et al., J Bacteriol 1994, 176(18):5718-5728; Treadway et al., Appl Microbiol Biotechnol 1999, 51(6):786-793; Moore et al., Plasmid 2003, 49(2):160-168; Rauzier et al., Gene 1988, 71(2):315-321; Stolt et al., Nucleic Acids Res 1997, 25(19):3840-3846; Ankri et al., FEMS Microbiology Letters 1996, 140(2-3):247-251; Stolt et al., Microbiology 1996, 142(10):2795-2802; Nesvera et al., J Bacteriol 1997, 179(5):1525-1532; Horii et al., Mol Gen Genet 1988, 212:225-231; Janniere et al., Gene 1990, 87(1):53-61; Janniere et al., Plasmids. In: Bacillus subtilis and other gram-positive bacteria: biochemistry, physiology, and molecular genetics. Edited by Sonenshein A L, Hoch J A, Losick R. Washington, D.C.: American Society for Microbiology; 1993: 625-644; Ballester et al., J Bacteriol 1989, 171(5):2271-2277; Michel et al., EMBO J. 1986, 5(13):3691-3696; Leonhardt et al., Gene 1991, 103(1):107-111; Bron et al., Mol Gen Genet 1988, 211(1):186-189; Muth et al., Mol Gen Genet 1989, 219:341-348; Kornberg et al., DNA replication, 2nd/edn. New York: W.H. Freeman; 1992; Janniere et al., Plasmids. In: Bacillus subtilus and other Gram Positive Bacteria: Biochemistry, Physiology, and Molecular Genetics. Edited by Sonenshein A L, Hoch J A, Losick R. Washington, D.C.: ASM; 1993: 1050; Bruand et al., Proc Natl Acad Sci USA 1993, 90(24):11668-11672; del Solar et al., Microbiol Mol Biol Rev 1998, 62(2):434-464; Larkin et al., Applied aspects of Rhodococcus genetics. Antonie Van Leeuwenhoek 1998, 74(1-3):133-153; Stolt et al., J Bacteriol 1996, 178(23):6693-6700; Chattoraj et al., Mol Microbiol 2000, 37(3):467-476; Pansegrau et al., Prog Nucleic Acid Res Mol Biol 1996, 54:197-251; Lessard et al., Appl Microbiol Biotechnol 1999, 52(6):811-819; Miller J H: Experiments in molecular genetics. Cold Spring Harbor, N.Y.: Cold Spring Harbor Laboratory; 1972; Eisenstadt et al., American Society for Microbiology; 1994: 297-316; de Lorenzo et al., J Bacteriol 1990, 172(11):6568-6572; Fellay et al., Gene 1987, 52(2-3):147-154; Follettie et al., Journal of Bacteriology 1993, 175(13):4096-4103; Altschul S F et al. 1990 J Mol Biol 215:403-410; Chan E C 1993 Biochem. Biophys. Res. Com. 194:525-530; Debrock B et al. 1994 Appl Environ Microbiol 60:853-60; Duffner F M et al. 2000 Gene 256:215-221; Ellis L B M et al. 2003 Nucleic Acids Research 31:262-265; Ensley B D et al. 1983 Science 222:167-169; Gibson D T et al 1995 J Bacteriol 177:2615-2621; Grund A 1995 SIM News 45:59-63; Kulakov L A et al. 1998 Microbiol 144:955-963; Leloup L et al. 1997 Applied and Environmental Microbiology 63:2117-2123; Mueller J G et al. 1989 Environ Sci Technol 23:1197-1201; Stafford D E et al. 2001 Adv Biochem Eng Biotechnol 73:85-101; Stafford D E et al. 2002 PNAS 99:1801-1806; Takai S et al. 2000 Infection and Immunity 68: 6840-6847; Wackett L P et al. 1988 Appl Environ Microbiol 27:1360-1367; Warhurst A M et al. 1994 Crit Rev Biotechnol 14(1):29-73; U.S. Pat. No. 5,871,981; and U.S. Pat. No. 5,858,737.

1. A method for introducing a nucleic acid into a Rhodococcus bacterium, comprising: culturing a first strain of Rhodococcus bacterium transformed with a plasmid comprising a nucleic acid sequence that enables conjugative transfer of the plasmid with a second strain of Rhodococcus bacterium for a length of time under conditions such that conjugative transfer occurs between said first and second strains of bacterium, whereby said plasmid is transferred from said first strain of bacterium to said second strain of bacterium.

2. The method of claim 1, wherein said plasmid further comprises a gene construct.

3. The method of claim 2, wherein said gene construct encodes a protein to be expressed in said second strain of bacterium.

4. The method of claim 3, wherein said gene construct further comprises at least one promoter suitable for the expression of a gene in a Rhodococcus bacterium.

5. The method of claim 4, wherein said gene is expressed in said second strain of Rhodococcus bacterium following said transferring.

6. The method of claim 3, wherein said plasmid comprises a mutant gene.

7. The method of claim 3, wherein said plasmid comprises a fragment of a gene.

8. The method of claim 1, wherein said plasmid or a portion thereof is integrated into the genome of said second strain of bacterium.

9. The method of claim 8, wherein said integration occurs by homologous recombination.

10. The method of claim 8, wherein the replication of said plasmid is temperature sensitive.

11. The method of claim 10, wherein said plasmid further comprises (a) SEQ ID NO: 2; (b) a sequence that hybridizes with (a) under stringent hybridization conditions; and (c) a sequence that is complementary to (a) or (b).

12. The method of claim 10, further comprising the step of selecting a bacterium of said second strain that has integrated said plasmid or a portion thereof into its genome by elevating the temperature of the culture.

13. A method for disrupting or knocking out a gene in a Rhodococcus bacterium, comprising: culturing a first strain of Rhodococcus bacterium transformed with a plasmid comprising a nucleic acid sequence that enables conjugative transfer of the plasmid and a nucleic acid sequence that serves to disrupt or knock out said gene with a second strain of Rhodococcus bacterium for a length of time under conditions such that conjugative transfer occurs between said first and second strains of bacterium, whereby said plasmid is transferred from said first strain of bacterium to said second strain of bacterium and said sequence that serves to disrupt or knock out said gene is integrated into the genome of said second second strain of bacterium.

14. The method of claim 13, wherein said integration occurs by homologous recombination.

15. The method of claim 13, wherein said nucleic acid sequence that enables conjugative transfer of the plasmid is selected from the group consisting of: (a) a nucleic acid sequence comprising the region involved in conjugal transfer containing origin of Transfer (oriT) sequence; (b) a nucleic acid sequence comprising the origin of Transfer (oriT) sequence; (c) a nucleic acid sequence that hybridizes with (a) or (b) under stringent hybridization conditions; and (d) a nucleic acid sequence that is complementary to (a), (b) or (c).

16. A method for selecting from a culture a recipient Rhodococcus bacterium that has integrated a plasmid or a portion thereof into its genome comprising the use of temperature selection.

17. The method of claim 16, comprising conjugatively transferring from a donor Rhodococcus bacterium to a recipient Rhodococcus bacterium that can grow above a minimum temperature a plasmid that cannot replicate above said minimum temperature; elevating the temperature of the culture above said minimum temperature for a sufficient time to prevent replication of said plasmid and survival of said donor Rhodococcus bacterium, whereby only a recipient Rhodococcus bacterium that has incorporated said plasmid into its genome survives such temperature elevation.

18. The method of claim 17, wherein said plasmid comprises (a) the minimal replicon sequence; (b) a sequence that hybridizes with (a) under stringent hybridization conditions; and (c) a sequence that is complementary to (a) or (b).

19. The method of claim 17, wherein said minimum temperature in 30° C. and the temperature is elevated to 37° C.

20. The method of claim 18, wherein said donor Rhodococcus bacterium is of the strain Rhodococcus B264-1 and said recipient Rhodococcus bacterium is of the strain Rhodococcus I24.


SEQUENCE LISTING (21 sequences)

SEQ ID NO: 1 - DNA, 4970 bp, Rhodococcus sp.
gaggaacgtcgggcggtcgagcgcggcgtcgtcgcgtgtggagttgcgcgagcgggtggtcgaggtcgagaaggtggtgcatcggccggccacgacggccgcgtccatcgaccgcgtactcgacgacgatcaggcgctgcagaagctgttgaacacgctgcggcaccgcatcaaggaaggcacctaccggcccggcggtgacaggaacatgacgtgggtgctccggaggctcacggccccggtggccgatctgctcgcccagatccactcgagtgcaggcaccaccgcgtacatcgacccggcgcaggccgtgcaggcagtgctcggctcaccacgctcgtgccgcgcggtcctggaaggcctcgacacaatggcccgctccggagagctcgacggcggccagcacaccgccacactgacagccctcgaccggctcgtagctcacacccggcagcagagccgatagaaaacggagctgccgcggcaccgctgcgcgctgccttgccaccgaaacgatgtcccttacgacggggaccgagggagtgaccgcatgagtcttgatcagcagatagccgagctgcgcgagcagttgaacgccctcggccgccaggtgcgcgaggtgacagaggccgccgacgagcaggacaagcccgacgtcgtcgacgccctggccgacgtgcgcatgcacctcaacgacgccgccgccgtcctcggggacgtgatcgcatgagcgactggacgagctgggtctgtcccaggtcaccgacccagcaacacgtccccgctggcctaccgccacgtcacgcccaggtgagggacactgacccacgtgaccaacccagggcagacgttcaccgtgaccacggccgccaaggcgtgccaggtgtcgcggaagacgatcactcgacgccttgaccagctgcgagacggcggcgcgtacaaggatgcggccggcgcgtgggtgatcccgcttggttcgctcctccacgccggtctcagtccgggtaggccagcgtccccggatgctgtccacgtgtcccaggaggacacacagggacaggaggacagaggctggttcaccctcgagcaagtcgcggaattgcgtaagcgcgccgacgaggcggagtcacttcgccggcgggccgaggtcgcggaagccctggccacagagcgggaaagagtgatcgaggtgcaggcgcgcgccctgcggatgctcgaggccggccaacggccgaccgcgcaccaggaaacgacgtcagaggtttccgccacgccggccgatgtgccgccacgccgcggtccgctagaacgcctggtcggacggttcgggctgtagcgtttggcaaggtcaagccagcgccgaccggggcaattggtagagcaagtgactcttaatcactgggtcatatggcgagacccatggcgattatctgggaggaagacgcggtcggggacttctgtttcagcgccgtcgaagtgcctctgggatgggttgtttgtggaccgtagtcagctggtcgaaggctgcgccacagacggggcaaaagcctttgatgcgcgcgtggccgccgacctcgacatccgttggcccaccgagccattgctcgtttgaacaggtctcacagaccatcagggtcgcgcgtgcaacgcacccgcacggcagggctgtcccaagggggacgggctggtagtggacgtcatcgcagctgatactcatcgctcgattcctgccagggcgtgatggacagtgccgaccgagcatcccagctcattggcgatagctctcatggacatgccagtaccgcgtagctcccgtatcttctcgcgccgttgctgtgcccgggacaggtaggcctctcgcggctctgatgtccaccggatgatgctggccgtcgatacgcctgtgcgttcggcgagctcccgcactgttatcccgttgcgaggcagacgctcagtcatgtgtggcctcctcaattgccagtcgcacctgatccagtttggtgctgcgtgcacgccgggcctggccgctggcgacgccgcctttgcggccacgggcggattggatggtcgagagtgtggcttcgtagacggcgataccgtctttccagatgcgggatcgcgtggtgatccatcggtgaatgctggcggcgatggcgcgggcttcagagggtgggagttcctcggtgtagctggcgttgagggcggtgacttcgcggtggagggcgagttcgaggccggcgctgtcatgagtgggcaggtagttgcgcatgaggccggggcgggtggcccaggtgcgagcggcgtggaagattgcgcagttgcggccgagtcctatgggggcgtcacggtgggccttggtgtgctgccagcgcggggagggcatgtgtgtgccgagttcggcctcgagctcggcgaggctgcgcggttcggtgtggatccagtgcgtgtcccagcctgagtgtgtggggttcttggtcatcaggcctgagtagccgatgtccccttcgacggcccggcgtagcccttcggtaacggcggcggcgtacgcgaggggcttccgtcgggcgtactcggtgcgggtgaacggctcgcgcagccaccagtgcaggtgcgcatgcccgttgctgctgttttcgatcacggcgttcggcatagggtgattgcctgccgctgacagtgcgcgcagggcggagtcggggtggtcgacgtcgacgacgagcagattgctcagtgtctgcgggttggcctcgatgtagcgccgctcaagtgcggcagggcggcgcattcgatagaccccctcaaggaagtcgtttgttgccaaaggccagaacggtagccacatttgctcccagtccccaccttcccgcaccccgtgtgtgtccatgctggtgaccgtcgcatgctctcgaacgaaacctgggcatttccctcggtgtgttcaaagttaatcgtgaagccaagtcagatgtttcggtgtcgccaggcgcccctcggtgtgttcaaagttaatcgtgaagccaagtcagatgtttcggtacgcccctggggaaaccccttcaagtccggcactcccaatgcctcatcgtgcgcatgcaggtttgaaacggccggcggcttaatttcgtcgcgcgaaaacctctccacggtctctgaagttcagcaagaggtgtgcctgaatttcagtgcgcccggactcgcaacgaagtgcccgcttgctgatttgtcggcgactcagtcggtgccatgtacggacgcgtgacgtttcaccacggtgcgggcgagatcagcgatcttgacgttcatgttctgtgaggttcgcctgagaatgtcgaaggcgtcctcagacgacacgtcgtgcagtgccatcagcagaccgatcgccttaccgatctcgcgattgctctccagcccgcgccgcagggtgactgcctcttctccccaggtgatggcagccaccgtcacggctgcgaatgacgcgagcacggcagcgggtcctgcggcctcagtcgtgaacgcccccggagtgtcactgaagagattcagtgctccaactttgtggccgtcgacgagcagacgaaatcccatgacgccacgaaccggagtttgtgcgagcacctggcgcgccagccgcggccagccggtgggtgtggcgaggtcgggatcgagttgtgcggcttcctcctctatcgcgtcgaggcacggtccctccccggtctggcgctcgagatcgtctaccagccgcgcgatgtcgtccgatgcggccaccgtcacgggtaaatcccggccacgcaacatcaggcttgcgtggtcacagccgggaaccagcagggtggcggcgacacagaccgccgcgtaaacctcggacggctcggaaccgttgtagacaatgtcggcgagcgcggcgaatacggtccccggatccgacttgctgggggtagctgcgctctgcgcgtccgtcatgacgttcctgctttcaagcccctctgggctgcgtcaaacgctacgcggtggaactgctcgggagtcatcccagatggtggagtgaaaggcgtgcgtttggcatcggccgaggcccgataaccgtcgtccgcgcggccgcagagcgctgagcaagataggcacttacctcacgtaagtgaggagtgatgggtgcgcgagcgatcgcgcaccggagacaccgggtgaaggctgggtccggtagcgcagggacagcacgcggcgcagcctgactcgctaccttgctgactcgctaccttgctgactcgctaccttgctgactcgctaccttgctgactcgctacctacgcggcgcagcaagatggagacttaggtgccctaagtcgcagcccgcattcgcgggatccctgtcggggcgctgcgctgggcggtgaaataccaaccttgcggttcctgggaacagctaaaccgaacggtgtcgcttcggcagtcggggatgacgacgccaggtgcgctgggggctacaaaggaacgagatgctcgctggtgttaccgaggtggtgtgccaacgagaatccaacttgccgcagcgtttggtgtttttgggggggaagcggatctcccgatgaggggcactgccacgcacactgaggggcgatccgcatgggtgtttcacgggcacggtcacttgcgtactgggagcagcacgtctggatcgaaacgcacgaccacatatccccgggccggtaccaagtgccggccgagaaacgtgcatctcccgcggacaccgtgtcggcacctgagaaccaagccgagtaattccacgacgcaacagatccagatccgacacacctcttgcactcaggagtgaatgtcggggcattttcgcgcgacgaaattaggatcgcggctatgacgacgccgcgatgcccccgctgtgaccgcgagatcgagagcaacaccagcgcactgcacggtcgcggccggccgcggaagtggtgctcggacgagtg

SEQ ID NO: 2 - DNA, 1341 bp, Rhodococcus sp.
cgtcatcgcagctgatactcatcgctcgattcctgccagggcgtgatggacagtgccgaccgagcatcccagctcattggcgatagctctcatggacatgccagtaccgcgtagctcccgtatcttctcgcgccgttgctgtgcccgggacaggtaggcctctcgcggctctgatgtccaccggatgatgctggccgtcgatacgcctgtgcgttcggcgagctcccgcactgttatcccgttgcgaggcagacgctcagtcatgtgtggcctcctcaattgccagtcgcacctgatccagtttggtgctgcgtgcacgccgggcctggccgctggcgacgccgcctttgcggccacgggcggattggatggtcgagagtgtggcttcgtagacggcgataccgtctttccagatgcgggatcgcgtggtgatccatcggtgaatgctggcggcgatggcgcgggcttcagagggtgggagttcctcggtgtagctggcgttgagggcggtgacttcgcggtggagggcgagttcgaggccggcgctgtcatgagtgggcaggtagttgcgcatgaggccggggcgggtggcccaggtgcgagcggcgtggaagattgcgcagttgcggccgagtcctatgggggcgtcacggtgggccttggtgtgctgccagcgcggggagggcatgtgtgtgccgagttcggcctcgagctcggcgaggctgcgcggttcggtgtggatccagtgcgtgtcccagcctgagtgtgtggggttcttggtcatcaggcctgagtagccgatgtccccttcgacggcccggcgtagcccttcggtaacggcggcggcgtacgcgaggggcttccgtcgggcgtactcggtgcgggtgaacggctcgcgcagccaccagtgcaggtgcgcatgcccgttgctgctgttttcgatcacggcgttcggcatagggtgattgcctgccgctgacagtgcgcgcagggcggagtcggggtggtcgacgtcgacgacgagcagattgctcagtgtctgcgggttggcctcgatgtagcgccgctcaagtgcggcagggcggcgcattcgatagaccccctcaaggaagtcgtttgttgccaaaggccagaacggtagccacatttgctcccagtccccaccttcccgcaccccgtgtgtgtccatgctggtgaccgtcgcatgctctcgaacgaaacctgggcatttccctcggtgtgttcaaagttaatcgtgaagccaagtcagatgtttcggtgtcgccaggcgcccctcggtgtgttcaaagttaatcgtgaagccaagtcagatgtttcggt

SEQ ID NO: 3 - DNA, 744 bp, Rhodococcus sp.
ctcgagatcgtctaccagccgcgcgatgtcgtccgatgcggccaccgtcacgggtaaatcccggccacgcaacatcaggcttgcgtggtcacagccgggaaccagcagggtggcggcgacacagaccgccgcgtaaacctcggacggctcggaaccgttgtagacaatgtcggcgagcgcggcgaatacggtccccggatccgacttgctgggggtagctgcgctctgcgcgtccgtcatgacgttcctgctttcaagcccctctgggctgcgtcaaacgctacgcggtggaactgctcgggagtcatcccagatggtggagtgaaaggcgtgcgtttggcatcggccgaggcccgataaccgtcgtccgcgcggccgcagagcgctgagcaagataggcacttacctcacgtaagtgaggagtgatgggtgcgcgagcgatcgcgcaccggagacaccgggtgaaggctgggtccggtagcgcagggacagcacgcggcgcagcctgactcgctaccttgctgactcgctaccttgctgactcgctaccttgctgactcgctaccttgctgactcgctacctacgcggcgcagcaagatggagacttaggtgccctaagtcgcagcccgcattcgcgggatccctgtcggggcgctgcgctgggcggtgaaataccaaccttgcggttcctgggaacagctaaaccgaacggtgtcgcttcggcagtcggggatgacgacgcc

SEQ ID NO: 4 - DNA, 91 bp, Rhodococcus sp.
ggcgcctggcgacaccgaaacatctgacttggcttcacgattaactttgaacacaccgagggaaatgcccaggtttcgttcgagagcatgc

SEQ ID NO: 5 - DNA, 933 bp, Rhodococcus sp.
atggacacacacggggtgcgggaaggtggggactgggagcaaatgtggctaccgttctggcctttggcaacaaacgacttccttgagggggtctatcgaatgcgccgccctgccgcacttgagcggcgctacatcgaggccaacccgcagacactgagcaatctgctcgtcgtcgacgtcgaccaccccgactccgccctgcgcgcactgtcagcggcaggcaatcaccctatgccgaacgccgtgatcgaaaacagcagcaacgggcatgcgcacctgcactggtggctgcgcgagccgttcacccgcaccgagtacgcccgacggaagcccctcgcgtacgccgccgccgttaccgaagggctacgccgggccgtcgaaggggacatcggctactcaggcctgatgaccaagaaccccacacactcaggctgggacacgcactggatccacaccgaaccgcgcagcctcgccgagctcgaggccgaactcggcacacacatgccctccccgcgctggcagcacaccaaggcccaccgtgacgcccccataggactcggccgcaactgcgcaatcttccacgccgctcgcacctgggccacccgccccggcctcatgcgcaactacctgcccactcatgacagcgccggcctcgaactcgccctccaccgcgaagtcaccgccctcaacgccagctacaccgaggaactcccaccctctgaagcccgcgccatcgccgccagcattcaccgatggatcaccacgcgatcccgcatctggaaagacggtatcgccgtctacgaagccacactctcgaccatccaatccgcccgtggccgcaaaggcggcgtcgccagcggccaggcccggcgtgcacgcagcaccaaactggatcaggtgcgactggcaattgaggaggccacacatgactga

SEQ ID NO: 6 - DNA, 246 bp, Rhodococcus sp.
atgactgagcgtctgcctcgcaacgggataacagtgcgggagctcgccgaacgcacaggcgtatcgacggccagcatcatccggtggacatcagagccgcgagaggcctacctgtcccgggcacagcaacggcgcgagaagatacgggagctacgcggtactggcatgtccatgagagctatcgccaatgagctgggatgctcggtcggcactgtccatcacgccctggcaggaatcgagcgatga

SEQ ID NO: 7 - DNA, 78 bp, Rhodococcus sp.
ctgactcgctaccttgctgactcgctaccttgctgactcgctaccttgctgactcgctaccttgctgactcgctacct

SEQ ID NO: 8 - DNA, 110 bp, Rhodococcus sp.
ccctcggtgtgttcaaagttaatcgtgaagccaagtcagatgtttcggtgtcgccaggcgcccctcggtgtgttcaaagttaatcgtgaagccaagtcagatgtttcggt

SEQ ID NO: 9 - DNA, 27 bp, Artificial Sequence
  Description of Artificial Sequence Synthetic primer
ggaattcgctgtcatgagtgggcaggt

SEQ ID NO: 10 - DNA, 30 bp, Artificial Sequence
  Description of Artificial Sequence Synthetic primer
cctgcagaaatgcccaggtttcgttcgaga

SEQ ID NO: 11 - DNA, 20 bp, Artificial Sequence
  Description of Artificial Sequence Synthetic primer
cacacctaaactgacatgct

SEQ ID NO: 12 - DNA, 18 bp, Artificial Sequence
  Description of Artificial Sequence Synthetic primer
gcaatatggtcttcgcct

SEQ ID NO: 13 - DNA, 20 bp, Artificial Sequence
  Description of Artificial Sequence Synthetic primer
cgagatcgagagcaacacca

SEQ ID NO: 14 - DNA, 19 bp, Artificial Sequence
  Description of Artificial Sequence Synthetic primer
cgttgtaaaacgacggcca

SEQ ID NO: 15 - DNA, 36 bp, Artificial Sequence
  Description of Artificial Sequence Synthetic primer
aaaaaacatatgagcacatcgacgtcgacgacgacc

SEQ ID NO: 16 - DNA, 35 bp, Artificial Sequence
  Description of Artificial Sequence Synthetic primer
aaaaggatcctcagccggcgagggtgagccggccg

SEQ ID NO: 17 - DNA, 20 bp, Artificial Sequence
  Description of Artificial Sequence Synthetic primer
gcaaggcgattaagttgggt

SEQ ID NO: 18 - DNA, 22 bp, Artificial Sequence
  Description of Artificial Sequence Synthetic primer
cgaagaccatattgctcaccga

SEQ ID NO: 19 - DNA, 20 bp, Artificial Sequence
  Description of Artificial Sequence Synthetic primer
ggactcgaagatcatctcgt

SEQ ID NO: 20 - DNA, 19 bp, Artificial Sequence
  Description of Artificial Sequence Synthetic primer
cgtactggttgaactgggt

SEQ ID NO: 21 - DNA, 21 bp, Artificial Sequence
  Description of Artificial Sequence Synthetic primer
ggagaagatcgtcgatgtcgt